SEQUENCE LISTING (39 sequences)

SEQ ID NO: 1 - DNA, 807 bp, Escherichia coli
  source 1..807: mol_type = unassigned DNA
atgcaggtcgacttgctgggttcggcgcaaagtgcccatgcgcttcacctgttccaccagcattccccactcgtacactgtatgaccaatgatgtagtgcagaccttcactgcgaatacactattggcccttggggcatccccggcgatggtcattgaaactgaggaagcttcacagttcgcagcgatcgcatccgctctactcattaacgtcggcacactaacacagcccagggcccaggctatgcgagcggctgttgaacaagccaagtcatctcaaacgccgtggactcttgaccctgttgccgtcggcgccctggattatagacgtcacttttgtcatgaattgttatcttttaaacccgccgcaatccgcggaaacgcatcggagatcatggctctggctggaatagctaatggcgggaggggggtggatactaccgacgccgctgcgaatgcgataccggcagctcaaaccttggcgcgggagacgggtgcaatcgtcgtcgtaactggagaaatggattacgtgacagacggtcaccgtattattggtatacatggtggggatccattaatgacgaaggtggttgggaccggctgcgccctaagtgcagtagtagccgcttgctgtgcattacctggcgatacgcttgaaaacgtggcaagcgcatgccattggatgaaacaagcaggagagcgagcggttgctcggagcgagggaccagggtcgttcgttccccattttctcgacgccttatggcaactcacgcaggaggtgcaagcgcaccaccaccaccatcattga

SEQ ID NO: 2 - DNA, 942 bp, Abies grandis
  source 1..942: mol_type = other DNA
atgtccgaggctgcagatgtagagcgtgtttatgccgcgatggaagaagccgccggtctattgggggttgcctgtgctcgggataaaatctatcccctcctgagtacatttcaggataccctcgtagagggaggctcggttgtagtgttttctatggcaagtggcaggcatagtactgagctcgattttagcatatcggtacccacctcgcatggtgacccgtatgcgacggtcgtggaaaaggggctctttcctgcaaccggacacccggtggatgacctattagcggacacgcaaaaacatctgccggtctctatgttcgccattgacggtgaagtcacaggaggtttcaagaagacttatgcattcttcccaacggacaatatgcccggagttgcggaacttagtgcaataccatcaatgccacctgcggtggcagaaaacgctgagctttttgcaaggtatggcttggacaaagtgcagatgacaagcatggattacaagaagcgtcaggtgaatctatatttctcagagttgtcagctcaaaccctagaggctgaatctgttcttgctctggttcgagaattagggttgcatgtacctaacgagttaggcctaaagttttgcaaacgctcattctcggtctacccaacgttaaattgggaaacagggaaaattgatcgcctgtgttttgcggtaataagcaatgatcctaccctcgtcccgtcctccgatgagggcgacatcgagaaattccataactatgcgactaaagctccctacgcctacgtaggtgagaagcgaacactggtttacgggttgacgttatctcctaaagaagaatactacaagcttagcgccgcataccacattactgacgtccaaagaggactcttaaaagccttcgactcccttgaggaccaccaccatcatcaccactga

SEQ ID NO: 3 - DNA, 567 bp, Streptomyces alanosinicus
  source 1..567: mol_type = other DNA
atgcagacggaacatgtgatattgctcaatgcccaaggagttccaaccggaacccttgagaagtacgcggcacataccgctgacacgaggctccatctagcgttttcctcctggctattcaacgcaaaggggcaactcttggtaactagacgagcccttagcaagaaagcgtggccaggtgtctggacaaattcagtttgtgggcacccacagcttggtgagagtaacgaggatgccgttatcagacggtgtcgatacgagctgggcgtcgaaatcacacccccggagagcatttatcccgattttcgctatcgggccactgacccttctggcattgtggaaaacgaggtatgcccggtatttgcggcacgtacgacctcagctctgcagataaatgacgatgaagtcatggactatcaatggtgcgatttagcggatgtgttgcatggtattgacgccactccttgggcattctcgccttggatggtaatgcaagctacgaatcgtgaagctaggaaacgcttatctgcattcacacagctaaaacaccatcaccaccaccactga

SEQ ID NO: 4 - DNA, 915 bp, Methanothermobacter thermautotrophicus
  source 1..915: mol_type = other DNA
atggttgaatttgattttaacaagtacatggactcaaaagcaatgacagtgaatgaggctctgaacaaagcaataccgctcagatatccgcagaaaatatatgaaagcatgcgttactcactgcttgccgggggaaagcgcgtccgtccggtcctgtgtatcgccgcatgtgaacttgttggtggtactgaggaactagcaatcccaacagcgtgcgcaattgagatgattcacacgatgtcgttgatgcatgatgacctaccatgcattgacaatgatgatcttagacggggtaaacccacaaaccataaaatttttggggaggatactgctgtgactgcgggtaacgcgttacacagctacgcgtttgaacatattgccgtctctaccagcaaaacggtaggcgccgaccggattcttcgaatggtgtcagagttgggtagagccacgggaagtgaaggcgtaatgggcggtcaaatggtcgatatagcgtctgagggagatccctccatagacttacaaacactagagtggatccatatccacaagactgcgatgctcctcgagtgtagtgttgtctgtggggccatcattgggggggctagtgaaatagttatagaacgggcacgcaggtacgctcgatgcgttggactcttattccaagtagtagacgacatactagatgtaaccaagtcctccgatgagctgggcaagaccgcgggcaaggacttaatctctgacaaagcaacgtatcctaagttaatgggccttgagaaagccaaggaattctcggacgaattgctaaatcgcgctaagggagaactctcgtgcttcgatccagtgaaagcggcccctttgctgggattggctgattatgtggctttcaggcagaatcaccatcatcaccaccattga

SEQ ID NO: 5 - DNA, 819 bp, Escherichia coli
  source 1..819: mol_type = other DNA
atgatcatccttaaactgggcggatcggtgatcacacgtaaggattcggaggagcccgctattgatcgggacaatttagagcgcattgcatccgagatcggtaatgcgagcccttcatctctcatgatagttcacggggcaggaagtttcggccacccattcgctggagaatacaggataggctctgaaatagagaacgaggaagatttacgaagacgtcgttttgggtttgccctgactcaaaattgggtgaaaaaattgaactcccacgtgtgcgacgcgctcctagcggaaggcattcctgctgtgagcatgcagccatcagcattcatacgcgcacatgcgggacggatctcacatgccgatatttcactgatacgatcttaccttgaggaagggatggtaccagttgtatatggggatgttgtccttgacagtgaccggcgcctcaaatttagcgtgattagcggcgatcagctaatcaatcacttttcgcttcgattaatgcctgagagggtaatattagggaccgacgtagatggtgtatatacgaggaaccccaagaagcatccggacgcccggctactagacgttattggcagtctcgacgacctcgagtcgctagatggtactctgaacactgatgtcaccggtggaatggtcggtaaaataagagaattgttgttgttagccgagaagggagtcgaatccgaaatcataaacgcggcagttccgggtaatattgaaagagctcttctgggcgaggaagtcagagggacgcgtattacaggaaagcatcatcaccaccatcaccattga

SEQ ID NO: 6 - DNA, 22 bp, synthetic construct
  source 1..22: mol_type = other DNA
ggatgcaatgcctgttcttttc

SEQ ID NO: 7 - DNA, 20 bp, synthetic construct
  source 1..20: mol_type = other DNA
agtttcaatgaccatcgccg

SEQ ID NO: 8 - DNA, 21 bp, synthetic construct
  source 1..21: mol_type = other DNA
ggaaagcatcatcaccaccat

SEQ ID NO: 9 - DNA, 20 bp, synthetic construct
  source 1..20: mol_type = other DNA
aacccaaaacacccttgagc

SEQ ID NO: 10 - RNA, 19 nt, synthetic construct
  source 1..19: mol_type = other RNA
gtaatgcgagcccttcatc

SEQ ID NO: 11 - RNA, 20 nt, synthetic construct
  source 1..20: mol_type = other RNA
cgccgctaatcacgctaaat

SEQ ID NO: 12 - RNA, 20 nt, synthetic construct
  source 1..20: mol_type = other RNA
aagtcatctcaaacgccgtg

SEQ ID NO: 13 - RNA, 20 nt, synthetic construct
  source 1..20: mol_type = other RNA
ctcgctctcctgcttgtttc

SEQ ID NO: 14 - RNA, 20 nt, synthetic construct
  source 1..20: mol_type = other RNA
acgtaggtgagaagcgaaca

SEQ ID NO: 15 - RNA, 20 nt, synthetic construct
  source 1..20: mol_type = other RNA
tcagtggtgatgatggtggt

SEQ ID NO: 16 - RNA, 20 nt, synthetic construct
  source 1..20: mol_type = other RNA
ggtcgttcgttccccatttt

SEQ ID NO: 17 - RNA, 20 nt, synthetic construct
  source 1..20: mol_type = other RNA
caatgatggtggtggtggtg

SEQ ID NO: 18 - RNA, 20 nt, synthetic construct
  source 1..20: mol_type = other RNA
tcatccttaaactgggcgga

SEQ ID NO: 19 - RNA, 20 nt, synthetic construct
  source 1..20: mol_type = other RNA
gatgaagggctcgcattacc

SEQ ID NO: 20 - RNA, 20 nt, synthetic construct
  source 1..20: mol_type = other RNA
ggcattgtggaaaacgaggt

SEQ ID NO: 21 - RNA, 20 nt, synthetic construct
  source 1..20: mol_type = other RNA
ccgctaaatcgcaccattga

SEQ ID NO: 22 - RNA, 20 nt, synthetic construct
  source 1..20: mol_type = other RNA
cgtctctaccagcaaaacgg

SEQ ID NO: 23 - RNA, 20 nt, synthetic construct
  source 1..20: mol_type = other RNA
cattacgccttcacttcccg

SEQ ID NO: 24 - DNA, 20 bp, synthetic construct
  source 1..20: mol_type = other DNA
agaggtggtagaacggcttc

SEQ ID NO: 25 - DNA, 20 bp, synthetic construct
  source 1..20: mol_type = other DNA
cggtcaagtcaacaacggag

SEQ ID NO: 26 - DNA, 20 bp, synthetic construct
  source 1..20: mol_type = other DNA
acacctacaattggagccca

SEQ ID NO: 27 - DNA, 21 bp, synthetic construct
  source 1..21: mol_type = other DNA
tgaaatgtactcaggagggga

SEQ ID NO: 28 - RNA, 20 nt, synthetic construct
  source 1..20: mol_type = other RNA
gcaaggtatggcttggacaa

SEQ ID NO: 29 - RNA, 20 nt, synthetic construct
  source 1..20: mol_type = other RNA
cgtcaacccgtaaaccagtg

SEQ ID NO: 30 - RNA, 20 nt, synthetic construct
  source 1..20: mol_type = other RNA
acgtaggtgagaagcgaaca

SEQ ID NO: 31 - RNA, 20 nt, synthetic construct
  source 1..20: mol_type = other RNA
ccttgcccttacaacggaaa

SEQ ID NO: 32 - RNA, 20 nt, synthetic construct
  source 1..20: mol_type = other RNA
taccgctcagttatccgcag

SEQ ID NO: 33 - RNA, 20 nt, synthetic construct
  source 1..20: mol_type = other RNA
cattacgccttcacttcccg

SEQ ID NO: 34 - RNA, 20 nt, synthetic construct
  source 1..20: mol_type = other RNA
tggctattcaacgcaaaggg

SEQ ID NO: 35 - RNA, 20 nt, synthetic construct
  source 1..20: mol_type = other RNA
gcattaccatccaaggcgag

SEQ ID NO: 36 - DNA, 20 bp, synthetic construct
  source 1..20: mol_type = other DNA
ggcttgagtcctggctacat

SEQ ID NO: 37 - DNA, 18 bp, synthetic construct
  source 1..18: mol_type = other DNA
ccctttgcgttgaatagc

SEQ ID NO: 38 - DNA, 21 bp, synthetic construct
  source 1..21: mol_type = other DNA
tgtaaccaagtcctccgatga

SEQ ID NO: 39 - DNA, 20 bp, synthetic construct
  source 1..20: mol_type = other DNA
ggttgttctcacctgcttcg

(19) United States
(12) Patent Application Publication
(10) Pub. No.: US 2024/0318211 A1
(43) Pub. Date: Sep. 26, 2024

(54) RECOMBINANT PRODUCTION OF CANNABINOIDS

(71) Applicant: 1000128725 Ontario Inc., Markham (CA)

(72) Inventors: Hamed Hasheminejad, Maple (CA); Mahdi Shahsavand, Richmond Hill (CA)

(21) Appl. No.: 18/587,332

(22) Filed: Feb. 26, 2024

Related U.S. Application Data
(60) Provisional application No. 63/486,818, filed on Feb. 24, 2023.

(51) Int. Cl. C12P 17/06; C12N 9/10; C12N 9/12; C12N 9/90; C12P 7/02

(52) U.S. Cl. CPC C12P 17/06; C12N 9/1085; C12N 9/1205; C12N 9/1229; C12N 9/90; C12P 7/02; C12Y 205/01001; C12Y 205/01039; C12Y 207/0105; C12Y 207/04026; C12Y 503/03002

(57) ABSTRACT

Ways of making and using a recombinant organism configured to produce a cannabinoid are provided. The recombinant organism can include a eukaryotic microorganism expressing a recombinant construct including a geranyl diphosphate synthase (GPPS2), an isopentenyl diphosphate isomerase (IDI), an isopentenyl phosphate kinase (IPK), and a 5-(hydroxyethyl)-methyl thiazole kinase (ThiM). A cannabinoid can be produced by a process that includes growing the recombinant organism configured to produce the cannabinoid in a growth medium and separating the cannabinoid from the recombinant organism and the growth medium. A biosynthetic system for producing a cannabinoid is provided that includes a bioreactor, the recombinant organism configured to produce the cannabinoid, and a growth medium for the recombinant organism.

### CROSS-REFERENCE TO RELATED APPLICATIONS

This application claims the benefit of U.S. Provisional Application No. 63/486,818, filed on Feb. 24, 2023. The entire disclosure of the above application is incorporated herein by reference.

### FIELD

The present technology relates to the production of cannabinoids, including the biosynthesis of cannabinoids using recombinant organisms.

### SEQUENCE LISTING

A sequence listing is submitted electronically herewith and is incorporated herein by reference (filename: 72646-1 Sequence Listing 02-24-2024.xml, date created: Feb. 20, 2024, file size 39 kilobytes). The Sequence Listing submitted is part of the specification and is herein incorporated by reference in its entirety.

### INTRODUCTION

This section provides background information related to the present disclosure which is not necessarily prior art.

Cannabinoids include a diverse class of chemical compounds that act on cellular cannabinoid receptors, and which can alter neurotransmitter release in the brain. Cannabinoid ligands for cannabinoid receptors include endocannabinoids (produced naturally in the body by animals), phytocannabinoids (found in cannabis and some other plants), and synthetic cannabinoids (manufactured artificially, including chemical modification of naturally occurring cannabinoids). Notable phytocannabinoids include tetrahydrocannabinol, the primary psychoactive compound in cannabis, and cannabidiol, which is another major cannabinoid constituent of the cannabis plant. There are at least one hundred thirteen (113) different cannabinoids found in cannabis, and these cannabinoids can exhibit varied effects individually and in combination.

Examples of cannabinoids include those defined by the following classes of compounds: cannabigerol-type, cannabichromene-type, cannabidiol-type, tetrahydrocannabinol- and cannabinol-type, cannabielsoin-type, iso-tetrahydrocannabinol-type, cannabicyclol-type, and cannabicitran-type. All classes are derived from cannabigerol-type (CBG) compounds and differ mainly in the way this precursor is cyclized, where the cannabinoids are derived from their respective 2-carboxylic acids (2-COOH) by decarboxylation; e.g., catalyzed by heat, light, or alkaline conditions. A list of particular cannabinoids includes tetrahydrocannabinol (THC), tetrahydrocannabinolic acid (THCA), cannabidiol (CB)), cannabidiolic acid (CBDA), cannabinol (CBN), cannabigerol (CBG), cannabichromene (CBC), cannabicyclol (CBL), cannabivarin (CBV), tetrabydrocannabivarin (TI-CV), cannabidivarin (CBDV), cannabichromevarin (CBCV), cannabigerovarin (CBGV), cannabigerol monomethyl ether (CBGM), cannabielsoin (CBE), and cannabicitran (CBT).

Certain cannabinoids exhibit antioxidant and neuroprotective properties. These effects appear to take place through the triggering of the cannabinoid receptors in the endocannabinoid system. Cannabinoids may be potential therapeutic agents for the treatment of oxidative neurological disorders, such as cerebral ischaemia. As described in U.S. Pat. No. 6,630,507 to Hampson et al., which is incorporated herein by reference, cannabinoids have various antioxidant properties that make cannabinoids useful in the treatment and prophylaxis of a wide variety of oxidation associated diseases, such as ischemic, age-related, inflammatory, and autoimmune diseases. Cannabinoids may also have particular application as neuroprotectants, for example, in limiting neurological damage following ischemic insults, such as stroke and trauma, or in the treatment of neurodegenerative diseases, such as Alzheimer's disease, Parkinson's disease, and HIV dementia.

One source of phytocannabinoids includes cannabis, which is a genus of flowering plants in the family Cannabaceae, where the genus includes the species Cannabis sativa, Cannabis indica, and Cannabis ruderalis. The genus is indigenous to and originates from central Asia. The cannabis plant is also known as hemp, although this term is often used to refer only to varieties of cannabis cultivated for non-drug use. Cannabis has long been used for production of hemp fiber, hemp seeds and their oils, hemp leaves for use as vegetables and as juice, medicinal purposes, and as a recreational drug. Some cannabis strains have been selectively bred to produce minimal levels of tetrahydrocannabinol, the principal psychoactive constituent. Medical cannabis (or medical marijuana) often refers to the use of cannabis and its constituent cannabinoids to treat disease or alleviate certain symptoms. For example, cannabis can be used to reduce nausea and vomiting during chemotherapy, to improve appetite in people with HIV/AIDS, and to treat chronic pain and muscle spasms. Cannabinoids derived from cannabis are also under investigation for their potential to affect stroke. Certain applications of cannabis and cannabinoids include the promotion of relaxation, alleviation of anxiety, treatment of post-traumatic stress disorder (PTSD), and mitigation of pain symptoms.

It would be desirable to biosynthetically produce certain cannabinoids in order to optimize productivity, control purity, improve scalability, and maximize cost effectiveness in obtaining and using cannabinoids.

### SUMMARY

The present technology includes ways of making and using recombinant micro microorganisms configured to produce one or more cannabinoids. The recombinant microorganism can include a eukaryotic microorganism expressing a recombinant construct including a geranyl diphosphate synthase (GPPS2), an isopentenyl diphosphate isomerase (IDI), an isopentenyl phosphate kinase (IPK), and a 5-(hydroxyethyl)-methyl thiazole kinase (ThiM). Aspects of the eukaryotic organism can include where the eukaryotic organism is a member of the fungus kingdom, such as a methylotrophic yeast. A particular example of a recombinant microorganism includes Pichia pastoris, also known by the genus Komagataella. Aspects of the recombinant construct include where the geranyl diphosphate synthase (GPPS2) is derived from Abies grandis, where the isopentenyl diphosphate isomerase (IDI) is derived from E. coli, where the isopentenyl phosphate kinase (IPK) is derived from Methanothermobacter thermautotrophicus (also referred to herein as MtIPK), and/or where the 5-(hydroxyethyl)-methyl thiazole kinase (ThiM) is derived from E. coli. The recombinant construct can further include prenyltransferase (NphB), including where the prenyltransferase (NphB) is derived from Streptomyces bacteria.

Various ways can be used to incorporate the recombinant construct into the microorganism, including where the recombinant construct is configured as a single recombinant element or multiple recombinant elements. The recombinant construct can also be incorporated in a single event or where different portions of the recombinant construct are incorporated in a single event or multiple events, including multiple sequential events. The recombinant construct can be incorporated into the genomic DNA of the recombinant microorganism, for example, using CRISPR. Other aspects include where the recombinant organism includes a Ku70 gene knockout.

Recombinant organisms configured to produce one or more cannabinoids can be used in various ways. One or more cannabinoids can be produced by a process that includes growing the recombinant organism configured to produce the cannabinoid in a growth medium and separating the cannabinoid from the recombinant microorganism and the growth medium. Similarly, a biosynthetic system for producing a cannabinoid can be provided that includes a bioreactor, the recombinant organism configured to produce the cannabinoid, and a growth medium for the recombinant organism.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIGS. 1A, 1B, and 1C depict cannabinoid biosynthesis pathways in the cannabis plant including enzymes involved in the pathway steps.

FIG. 2 depicts plasmid extraction and linearization, shown from left to right are AOX1 plasmid extraction, FLD1 plasmid extraction, molecular marker, linearized FLD1, and linearized AOX1.

FIG. 3 depicts plasmid linearization by restriction enzymes, shown from left to right are molecular markers, FLD1 plasmid, AOX1 plasmid, GAP plasmid, IDI-GPPS2 plasmid, ThiM-mtiPK plasmid, and NphB plasmid.

FIG. 4 depicts plasmid extraction and linearization, shown from left to right are molecular markers, AOX1 plasmid extraction, and the fourth line is linearized AOX1 plasmid the fifth and seventh lines are linearized NphB, and the sixth line is NphB plasmid circular.

FIG. 5 depicts the cultivation of colonies and transformant colony verification, where No. 1: negative control (without plasmid+with hygromycin), No. 2: medium without any antibiotics (to identify the viability of Pichia cells), No. 3: colonies with CRISPR construct and without donor template, and No. 4: colonies with the CRISPR construct and with donor template.

FIG. 6 depicts DNA and RNA extraction from samples.

FIG. 7 depicts DNA verification with specific primers, where 1: 1 Kb molecular marker, 2: primer specific for NphB (1750 bp), 3: primer specific for mtipK (1590 bp), 4: primer specific for GPPS2 (1600 bp), 5: primer specific for NphB (1558 bp), 6: primer specific for IDI (1985 bp), and 7&8: primer specific for ThiM (2018 bp).

FIG. 8 depicts DNA verification by some specific primers, where 1) 1 kb molecular marker, 2) ΔKu70 with p1 primer, 3) NphB with p1 primer, 4) ΔKu70 with p2 primer, 5) NphB with p2 primer, 6&7) non-transgenic clones with a genome-specific primer which is designed from the Pichia genome, and 8) 100 bp molecular marker.

FIG. 9 depicts NphB verification with 3 primers. 1: molecular marker 1 kb 2: ΔKU70 strain with P1 primer, 3: transformed colony with P1 primer, 4: transformed colony with P2 primer 5: ΔKU70 strain with P2 primer, 6: transformed colony with P3 primer 7: ΔKU70 strain with P3 primer.

FIG. 10, verification of NphB gene with specific primers, where samples 3 & 4 are transformed and other colonies are not.

FIG. 11 depicts DNA verification to ThiM and mtiPK Genes.

FIG. 12 depicts the DNA verification of NphB, IDI & GPPS2.

FIG. 13 depicts semi-quantitative RT-PCR for RNA verification of NphB, where 1: the first-time course after induction (24 h) 2: the second time course after induction (48 h) 3: the third time course after induction (72 h) 4: Before induction 5: −KU70 strain before induction 6: −KU70 strain after induction.

FIG. 14 depicts DNA verification for IDI, GPPS2 genes, where 1: molecular marker, 2: ΔKU70, 3: negative control, 4, 5, 6, 7, 9, 10, and 11: colonies without gene insertion, and 8, 12, 13, 14, 15, 16, and 18: transformed colony with IDI and GPPS2 genes insertion.

FIG. 15 depicts RNA verification with semiquantitative RT PCR for IDI & GPPS2 Genes.

FIG. 16 depicts CRISPR colonies on YPDS medium with hygromycin antibiotics, where 1: Negative control without any CRISPR construct in the 200 ug/ml hygromycin 2: CRISPR construct without donor template in the 200 ug/ml hygromycin 3: positive control without CRISPR construct & hygromycin 4: CRISPR construct with donor template in the 200 ug/ml hygromycin.

FIGS. 17A, 17B, and 17C depict mtiPK PCR quantification with melt curve report and PCR baseline subtracted curve fit data.

FIGS. 18A, 18B, 18C, and 18D depict the GPPS PCR quantification report and PCR baseline subtracted curve fit data.

FIGS. 19A, 19B, 19C, and 19D depict ThiM PCR quantification report and PCR baseline subtracted curve fit data.

FIG. 20 depicts NphB PCR quantification report.

FIGS. 21 and 22 depict protein verification with SDS-PAGE, where M is: Molecular marker for protein, KU-70: is a negative control, P is: the sample before induction, P1: is the first-time course after induction, P2: is the second time course after induction, P3: third time course after induction, P4: fourth time course after induction.

FIG. 23 depicts a gene sequence for isopentenyl diphosphate isomerase (IDI) derived from E. coli following codon optimization for expression in Pichia.

FIG. 24 depicts a gene sequence for a geranyl diphosphate synthase (GPPS2) derived from Abies grandis.

FIG. 25 depicts a gene sequence for a prenyltransferase (NphB) derived from Streptomyces bacteria.

FIG. 26 depicts a gene sequence for an isopentenyl phosphate kinase (IPK) derived from Methanothermobacter thermautotrophicus, also referred to herein as MtIPK.

FIG. 27 depicts a gene sequence for a 5-(hydroxyethyl)-methyl thiazole kinase (ThiM) derived from E. coli.

FIG. 28 depicts a ThiM-MtIPK recombinant construct and associated primers.

FIG. 29 depicts an NphB recombinant construct and associated primers.

FIG. 30 depicts an IDI-GPPS2 recombinant construct and associated primers.

### DETAILED DESCRIPTION

The following description of the technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the object in the figures is turned over, elements described as “below” or “beneath” other elements or features would then be oriented “above” the other elements or features. Thus, the example term “below” can encompass both an orientation of above and below. The object may be otherwise oriented (rotated 90 degrees or at other orientations) and the spatially relative descriptors used herein interpreted accordingly.

The present technology provides a recombinant organism configured to produce a cannabinoid, where the recombinant organism can be used in certain processes and systems to biosynthetically produce certain cannabinoids in order to optimize productivity, control purity, improve scalability, and maximize cost-effectiveness in obtaining and using cannabinoids. A eukaryotic microorganism is provided that can express one or more recombinant constructs including a geranyl diphosphate synthase (GPPS2), an isopentenyl diphosphate isomerase (IDI), an isopentenyl phosphate kinase (IPK), and/or a 5-(hydroxyethyl)-methyl thiazole kinase (ThiM). The eukaryotic organism can include a member of the fungus kingdom, such as a methylotrophic yeast, where a particular example includes Pichia pastoris.

The recombinant construct can include the following aspects. The geranyl diphosphate synthase (GPPS2) can be derived from Abies grandis. The isopentenyl diphosphate isomerase (IDI) can be derived from E. coli. The isopentenyl phosphate kinase (IPK) can be derived from Methanothermobacter thermautotrophicus, referred to as MtIPK. The 5-(hydroxyethyl)-methyl thiazole kinase (ThiM) can be derived from E. coli. The recombinant construct can further include prenyltransferase (NphB), where the prenyltransferase (NphB) can be derived from Streptomyces. The recombinant construct can be incorporated into the genomic DNA of the recombinant organism, such as by using CRISPR gene editing, for example. The recombinant microorganism can include a Ku70 gene knockout, where disruption of the Ku70 gene can enable high frequency homologous recombination in a eukaryote recombinant organism.

Cannabinoids and ways of producing such can include a process of growing the recombinant organism configured to produce the cannabinoid in a growth medium and separating the cannabinoid from the recombinant organism and the growth medium. A biosynthetic system for producing the cannabinoid can include a bioreactor, the recombinant organism configured to produce the cannabinoid, and a growth medium for the recombinant organism. Alternatively, the recombinant organism can be grown and separated from the growth medium, where the recombination organism itself can be used as a source of the cannabinoid, or where the cannabinoid is only partially fractionated from the recombinant organism.

The subject cannabinoid can belong to a group of secondary metabolites that are produced in cannabis plants. Applications and uses for various cannabinoids, such as tetrahydrocannabinol (THC) and cannabidiol (CBD), have increased as certain cannabis products have been legalized for medical use for a variety of treatments, conditions, and diseases. Various types of cannabinoids can be produced using the present technology for such medical uses, where the cannabinoids can be further modified (e.g., chemically and/or enzymatically) to produce different types of cannabinoids, cannabinoid variants, and chemically modified cannabinoids. Cannabinoids produced biosynthetically using the present technology can be used in combination with each other, with one or more naturally sourced cannabinoids, with one or more modified cannabinoids, and/or with one or more chemically synthesized cannabinoids to provide certain combinations for various applications. For example, certain combinations of cannabinoids can provide a synergistic effect, referred to as an entourage effect, where the combination can modify overall activity or effects of one or more cannabinoids in the combination. Entourage effects can be provided by the combination of the cannabinoids as well as combinations of one or more cannabinoids with other compounds including terpenoids and flavonoids, for example.

The biosynthetic production of cannabinoids provided by the present technology realizes several benefits and advantages over the isolation of cannabinoids from cannabis plants. These benefits and advantages include at least the following: (1) access to minor cannabinoids that are currently not economically feasible to extract from plant sources and develop into drug candidates; (2) cost-savings relative to existing agricultural production methods (e.g., plant-grow-harvest-extract-purify); (3) increased yield of rare cannabinoid(s) with optimized fermentation, purification consistency, and quality control; (4) scalability to allow efficient and cost-effective supply as market demand increases; (5) the present technology can produce bio-identical cannabinoids to those found in nature; and (6) biosynthesis approaches provide a long-term sustainable option. The present technology accordingly provides a biosynthetic means to attain economical production of cannabinoids using a bioreactor.

Production of various types of cannabinoids, precursors to cannabinoids, and various cannabinoid intermediates are contemplated herein. Phytocannabinoids include meroterpenoids such as partial derivatives with a resorcinol core featuring a para position isopropyl, alkyl, or aralkyl sidechain that can be naturally produced as specialized metabolites in plants. Cannabis sativa is a known phytocannabinoid producer. For centuries, C. sativa has been used for several purposes, including as food and for medicinal purposes. Cultivation of C. Sativa allowed the production of various fiber products, and once the psychoactive property of the plant was recognized, the selection of desirable euphoric activity started. As a result of substantial breeding, several different C. sativa cultivars exist that are categorized by their morphology and chemical profiles. For example, hemp, which is a fiber-type cannabis, grows very tall and has a lower THC level, in comparison to marijuana which is a drug-type cannabis that grows shorter and has a higher THC level. According to the literature, more than 100 phytocannabinoids have been discovered in C. Sativa. These compounds can be separated into several categories based on their structures: CBG-type, CBC-type, CBD-type, THC-type, CBL-type, CBE-type, CBN-type, and sconmenhyl-type. THC is the one compound with the highest reported psychoactive property. All phytocannabinoids in C. Sativa can have an alkyl side chain attached to the central resorcinol core.

Phytocannabinoids are primarily synthesized and stored within glandular trichomes that are present on the female flowers and to a lower degree on the leaves. These trichomes contain resin storage cells where cannabinoids and terpenes build up to form a resin that is postulated to act as an anti-herbivory agent. The highest phytocannabinoid content is found at flower maturity, since as it grows the composition of cannabinoids and terpenes changes.

Due to the low existence of natural sources and strenuous isolation methods, natural cannabinoid products can be hard to attain in desired quantities. What is more, due to the complexity and hydrophobicity of cannabinoid compounds, feasible chemical synthesis can be hindered. Chemical synthesis can be enabled, however, by specialized organisms and developing solutions; e.g., synthesis of specific enzymes and reaction chambers. The introduction of enzymes, but also the translation of other natural solutions in technical applications can support chemical synthesis (Groger and Asian, 2011; Willrodt et al., 2015). The successful implementation of biocatalytic processes for chemical synthesis can be determined by the availability of a specific biocatalyst (Schmid et al., 2001) and its kinetic characteristics, but also the physical-chemical properties of the reactants, and the production technology.

The present technology can employ Pichia pastoris as a platform to produce cannabinoids. P. pastoris is a methylotrophic yeast, a heterotroph discovered in the 1960s. P. pastoris can survive and reproduce using different carbon sources, such as methanol, glucose, and glycerol. P. pastoris exists in two cell types, haploid and diploid cells, obtained through mitosis, sporulation, and meiosis, respectively. P. pastoris is a single-celled eukaryote. Proteins within P. pastoris can be studied and compared with other more complex eukaryotic species to understand their function and origin. Research into P. pastoris has developed this model organism into an advanced heterologous gene expression system that can be used to produce various heterologous proteins. Due to its ability to serve as a model organism for genetic studies and as a protein expression system, P. pastoris has become an important microbial strain for biological research and various recombinant gene expression applications. For example, P. pastoris can functionally process large molecular weight proteins, which can be useful in translation hosts.

Advantages related to growth and fermentation of P. pastoris as used in the present technology include the following aspects:
(1) Culture: P. pastoris can be grown on simple, inexpensive media with fast growth rates. P. pastoris is a methylotrophic yeast, where it can be grown with methanol as the sole energy source. P. pastoris can also be grown in shake flasks or fermenters, making it suitable for both small-scale and large-scale production.
(2) Growth: P. pastoris, like other widely used yeast models, has a relatively short lifespan and fast growth rate. P. pastoris can be grown in medium with high cell densities. These features are compatible with the expression of heterologous proteins and allow the production of higher yields.
(3) Production: The P. pastoris expression system is capable of producing high concentrations of heterologous proteins. P. pastoris is easy to manipulate genetically, and a wide range of vectors and strains are available. This eukaryote also can produce soluble and correctly folded recombinant proteins with post-translational modification (PTM), such as N-glycosylation. This is important where the target protein function may require eukaryotic processing that is not available in prokaryotes.

Based on the characteristics of P. pastoris, it can be used to produce various biopharmaceuticals and industrial enzymes. High cell density fermentation of P. pastoris can enable the production of large amounts of highly active and low-cost recombinant proteins. In addition, P. pastoris is capable of protein glycosylation for biotherapeutics. In the food industry, P. pastoris can be used to produce different kinds of enzymes as food additives with many functions. An example of P. pastoris includes the American Type Culture Collection (ATCC, Manassas, VA, USA) product referred to as Komagataella pastoris (Guilliermond) Yamada et al., 28485™, which has the strain designation: CBS 704 [CCRC 21531, DSMZ 70382, JCM 3650, NCYC 175, NRRL Y-1603]. Other P. pastoris strains, isolates, and related expression system platforms can be used in the present technology. Routine microbiological methods for growth, maintaining, and using P. pastoris include those described by Cregg, J. M., Barringer, K. J., Hessler, A. Y., & Madden, K. R. (1985). “Pichia pastoris as a host system for transformations,” Molecular and Cellular Biology. 5 (12): 3376-3385.

The present technology can employ various techniques to engineer and form the recombinant microorganism configured to produce the cannabinoid. One such methodology includes CRISPR technology for genome editing. Genome engineering by CRISPR/Cas9 (clustered regularly interspaced short palindromic repeats/CRISPR associated protein 9) has become a valuable tool in gene editing (Sander & Joung 2014; Wang et al. 2016; Doudna & Charpentier 2014). Aspects of using CRISPR can include the systems, methods, and compositions described in U.S. Pat. No. 8,697,359 to Zhang. CRISPR facilitates targeted, programmable genome modifications and provides major advantages over classical knock-out/knock-in approaches as well as alternative genome engineering strategies. Besides its potency in basic research for studying disease conditions (Xue et al. 2014; Matano et al. 2015; Wu et al. 2013; Baltimore et al. 2015), CRISPR/Cas9 is also used for the host (strain) engineering for biotechnological production processes (Roointan & Morowvat 2016; Krappmann 2016; Shen et al. 2017). Prior to CRISPR/Cas9, highly efficient genome manipulations could be difficult to achieve in certain organisms. Gene targeting with conventional knock-out/knock-in cassettes containing homologous sequences can depend on the organism's amenability for homologous recombination, where homology cassettes were predominantly integrated ectopically (Park et al. 2016; Weninger, Killinger, et al. 2015). In addition, the replacement of DNA regions or the insertion of markers (or other disruption cassettes) can change the context of the genome sequence and can influence neighboring genes and their expression. This can be relevant for maintaining the functional fidelity of tightly packed microbial genomes, where adjacent open reading frames are sometimes only separated by very short DNA stretches.

Genome engineering methods available prior to the advent of CRISPR/Cas9 such as Zinc-finger nucleases or TALENs (Gaj et al. 2013; Kim & Kim 2014; Weninger, Killinger, et al. 2015) require more laborious protein engineering to reprogram the targeting locus.

CRISPR/Cas9 allows to easily introduce targeted strand breaks in almost any desired genomic locus and can easily be reprogrammed. The present technology includes application of the CRISPR/Cas9 system to deliver recombinant DNA constructs into a eukaryotic microorganism, such as Pichia pastoris, where this methyltrophic yeast can be used to express various recombinant constructs. P. pastoris is also known by the genus Komagataella, which includes the species K. kurtzmanii, K. mondaviorum, K. pastoris, K. phaffii, K. populi, K. pseudopastoris, and K. ulmi. In particular, Komagataella phaffii (synonymous with Pichia pastoris) provides a host system for recombinant protein expression. Achieving targeted genetic modifications can be hindered by low frequencies of homologous recombination (HR). However, the present technology has implemented a CRISPR/Cas9 genome editing system for P. pastoris enabling gene knockouts based on indels (insertion, deletions) via nonhomologous end joining (NHEJ) at near 100% efficiency. Certain implementations can also include integrating homologous donor cassettes via HR.

In the present technology, CRISPR/Cas9 mediated integration of markerless donor cassettes can be achieved at efficiencies approaching 70-90% using a Ku70 deletion strain. The Ku70p is involved in NHEJ repair and the lack of the protein appears to favor repair via HR nearly exclusively. While an absolute number of transformants in a ΔKu70 strain can be reduced, virtually all surviving transformants show correct integration. In the wildtype strain, markerless donor cassette integration can also be improved up to 25-fold by placing an autonomously replicating sequence (ARS) on the donor cassette. Alternative strategies for improving donor cassette integration using a Cas9 nickase variant or reducing off-targeting associated toxicity using a high fidelity Cas9 variant did not produce the desired effects in P. pastoris. Furthermore, the present technology employs Cas9/gRNA expression plasmids with a geneticin (G418) resistance marker which provide a versatile tool for marker recycling. These CRISPR-Cas9 tools can be applied for modifying existing production strains and also enable markerless whole-genome modification in P. pastoris.

The present technology, in particular, provides a new pathway to produce cannabinoids, such as cannabigerolic acid (CBGA). In previous methods, genes that were involved in certain pathways had to be introduced to produce the cannabinoid. In the technology provided herein, genes from different pathways are combined to produce new avenues of biosynthetic production of the cannabinoid. One such example is the production of geranyl diphosphate (GPP), which is one of the most important substances in the cannabinoid biosynthesis pathway, where the present technology has combined isoprenoid alcohol production genes with a cannabinoid pathway gene to provide a new biosynthetic pathway by introducing these genes into the Pichia pastoris genome. By using such novel engineered biosynthetic pathways, the present technology can substantially increase the production of cannabinoids such as CBGA. Embodiments of the present technology can use CRISPR gene editing techniques to knock-in all genes involved in the present novel engineered biosynthetic pathways.

Certain cannabinoid biosynthesis pathways have been elucidated in the cannabis plant. In these natural biochemical pathways, the main substances being used in different stages can include hexanoic acid, malonyl co-enzyme A, and geranyl diphosphate (GPP) which produce the first cannabinoids as cannabigerolic acid (CBGA). The CBGA can then be converted into THC, CBD, and CBC by three different enzymes.

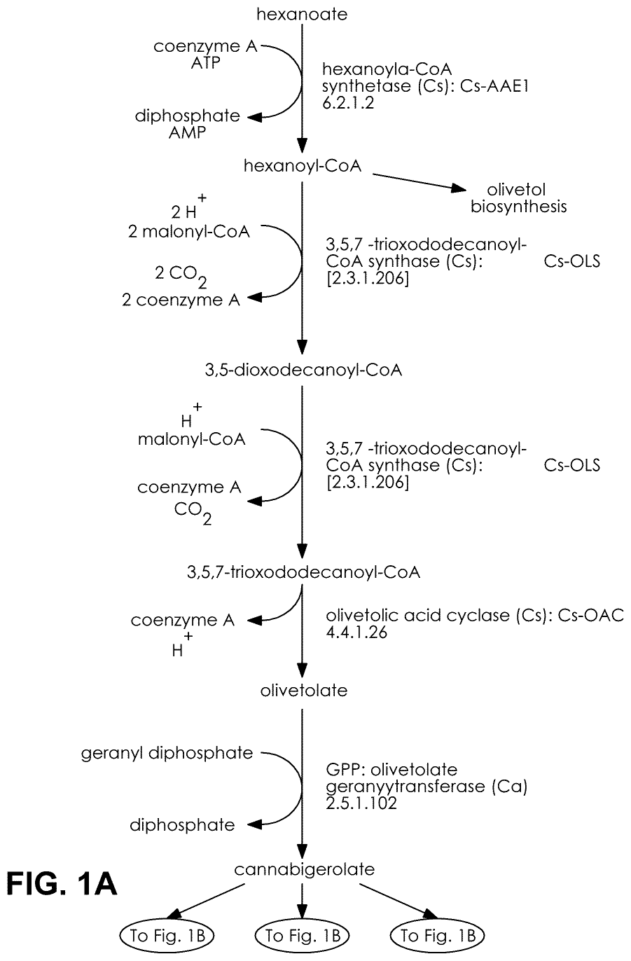

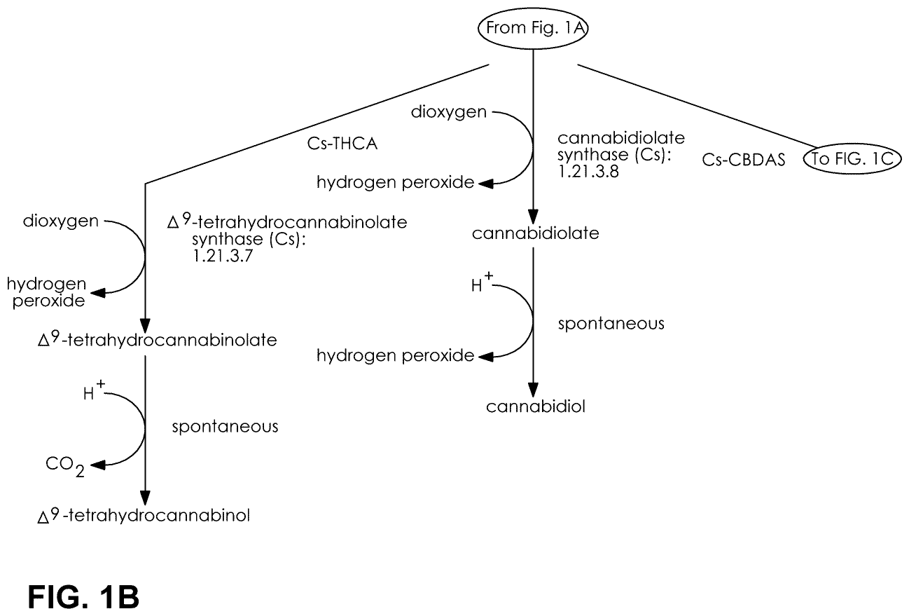

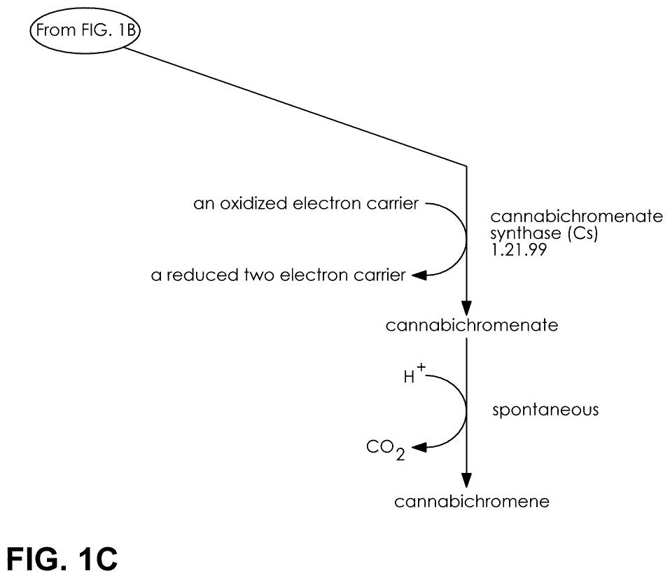

FIGS. 1A, 1i, and 1C depict cannabinoid biosynthetic pathways in the cannabis plant, including reaction components, reaction intermediates, cofactors, and enzymes involved in the pathways. As shown in the cannabinoid biosynthetic pathways, the important role of olivetolic acid and GPP as two main producers of cannabinoids can be appreciated. The GPP content for cannabinoid biosynthesis can be provided by the mevalonic acid (MVA) cycle or the methyl-erythritol-phosphate (MEP) cycle. In gram positive bacteria (e.g., enterococcus, staphylococcus, and streptococcus) as well as in most eukaryotic cells, such as plants, the MVA pathway for the production of GPP is required by the cells. In gram negative bacteria, however, often the MEP pathway is performed. Since the MVA and MEP pathways use high levels of energy due to the many enzymes involved in the process of GPP biosynthesis, an alternative synthetic pathway instead of a regular pathway for the production of GPP can be important.

In the production of cannabinoids in Pichia pastoris, the present technology can employ certain genes involved in biochemical pathways for cannabinoid biosynthesis. Certain genes can be obtained from cannabis and certain genes can be obtained from certain microorganisms, where these genes can then be transferred into Pichia pastoris to form the recombinant microorganism configured to produce a cannabinoid. To provide for GPP production, the present technology used alternative synthetic pathways instead of MEP and MVA. In particular, the present technology can utilize prenol as an inexpensive precursor material to biosynthetically produce GPP.

In engineering GPP production from prenol, the following four enzymes can be employed: geranyl diphosphate synthase (GPPS2), isopentenyl diphosphate isomerase (IDI), an isopentenyl phosphate kinase (IPK), and 5-(hydroxyethyl)-methyl thiazole kinase (ThiM). Genes for IDI and ThiM can originate from E. coli and the gene for IPK can originate from Methanothermobacter thermautotrophicus (referred to herein as MtIPK). Also, the GPPS2 gene can originate from Abies grandis. For GPP production, prenol can be converted into DMAP by the ThiM enzyme and then DMAP can be converted into DMAPP by the MtIPK enzyme. DMAPP is then converted into IPP by the IDI enzyme. Finally, the combination of IPP and DMAPP by the GPPS2 enzyme leads to GPP production.

In certain embodiments, certain compounds can be added to the growth medium to provide reactants for further cannabinoid production. For example, CBGA production can include where olivetolic acid is added to the Pichia pastoris growth medium, in order to combine with GPP to produce CBGA. In this pathway, the prenyltransferase (NphB) enzyme can be used, which can originate from Streptomyces CL-190.

Bioinformatic aspects related to the cannabinoid biosynthesis genes used in the present technology can include the following details. Upon selection and engineering of the biosynthetic pathways of the present technology, the structure of the genes and proteins can be identified and verified and any post-translational modifications (PTM) thereof can be considered. For these reasons, various gene data banks such as NCBI, Uniprot, Ensemble, Expassy, and Brenda can be used to identify analogous genes, derivatives, family members, and other sources having the selected enzymatic activities. As one example, after identification of a certain biosynthetic pathway and the enzymes involved therein, the sequences of the respective genes which produce the respective enzymes can be derived. This can include codon optimization, in certain instances. Additional gene information and protein structure can be obtained from NCBI and Ensemble data banks. By using protein data banks, such as Uniprot, Expassy, and the like, proteins produced by selected genes can be ascertained and modeled. This can include verification and optional inclusion or exclusion of certain signal sequences as well as PTM processes.

The present technology further ascertained aspects related to incorporation of recombinant constructs into the selected microorganism. Such aspects included the identification of the upstream sequence of promotors as a harbor-safe sequence to introduce genes in a given area. In order to analyze a given area, the NCBI database can be used to identify 100-200 base pairs upstream of a promoter region. Homology arms for donor templates can be designed from these regions. One consideration is that the given area (to introduce the genes into the genome) can be selected to minimize conflict with any previous gene terminator and any upcoming promoter. Another factor considered in the given area is the identification of binding of certain proteins (e.g., transcription factors) in the area. For example, in order to identify the reaction of the Pichia cells to manipulate selected genes (e.g., knock-out/knock-in), certain systems biology software such as Optflex3 can be used. In this software, after selection of the microorganism, it is possible to knock-out or knock-in certain genes and verify their effect on biomass value and growth.

The following aspects were used to produce a Ku70 knock-out strain in Pichia pastoris. CRISPR is a suitable technique for the process of gene knock-in and knock-out, and can be used in implementing the present technology. The CRISPR technique can employ two pathways—the first can be referred to as non-homologous end joining (NHEJ) and the second can be referred to as homologous recombination (HR). HR, in particular, can be used to knock-in the selected genes in the present technology.

In Pichia pastoris, unlike Saccharomyces cerevisiae, the process of HR is less utilized, where the Pichia strain was therefore manipulated to increase HR efficiency. The present technology therefore addresses the HR efficiency issue by silencing the Ku70 gene in Pichia. The Ku70 gene can be involved in NHEJ, and hence can be silenced to increase HR. A suitable microorganism for the HR processes described herein includes the GS115 strain of Pichia pastoris. The CRISPR technology can accordingly used to knock-in several genes. At the outset, however, the Ku70 gene can be knocked-out in the GS115 Pichia pastoris genome to improve and enhance homologous recombination. For this purpose, GS115 can be modified to delete, disrupt, or otherwise silence the Ku70 gene. The GS115 strain provides a significant increase with resepct to knock-in efficiency for genes of interest as it does not express the Ku70 gene. One source for the GS115 strain of Pichia pastoris yeast includes catalog number: C18100 from ThermoFisher Scientific (Waltham, MA, USA).

The following aspects were used in CRISPR construct production (e.g., promotor-gene-terminator) in order to knock-in selected genes into the Pichia pastoris genome. In order to increase HR efficiency in the GS115 Pichia strain, the Ku70 genes involved in NHJE pathway are silenced. In the next stage, two plasmids are used to knock-in genes: BB3nK and BB3cH. A donor sequence can be provided in the BB3nK plasmid, where the gRNA and Cas9 can be provided in BB3cH plasmid. The BB3nK plasmid includes an on sequence, an antibiotic resistance gene, and a donor or selected gene sequence. The BB3cH plasmid includes a hygromycin resistance gene, on sequence, and an automated replication sequence (ARS), a Cas9 sequence, and a SgRNA sequence. gRNA can be designed by ChopChop Software and can be tested with other software, such as Cas-offinder. After designing the gRNA, it can be produced synthetically and/or by recombinant techniques for incorporation into the plasmid constructs.

For designing the donor template, a gene data bank can be used to extract the sequence including the coding sequence (CDS), where two homology arms for the donor template can be designed. The resulting sequence constructs can be assembled using various software, such as provided by Integrated DNA Technologies, Inc. (Coralville, Iowa, USA). Donor templates along with gRNA can also be constructed using synthetic and/or recombinant techniques. Finally, after the construction, the respective constructs can be cloned into the BB3nK and BB3cH plasmids.

The following aspects can be used in Harbor Safe sequence development. Harbor safe is a predetermined location of the genome in which to introduce the desired genes. Selecting this region for the CRISPR knock-in can allow gene expression and protein synthesis to be influenced by this area. Accordingly, certain bioinformatic characterization can be performed on this region. Suitable regions to use as a harbor-safe sequence to introduce genes into the genome can include locations about 100 base pairs upstream of certain gene promoters, for two reasons: (1) these regions are active for biological aspects, (2) there typically are no essential genes located in this space. Suitable upstream regions of the Pichia pastoris genes can be accordingly identified, and upstream regions of the AOX1, FLD1 and GAP promoter regions can be selected as harbor-safe sequences.

Construction of the SgRNA (ribozyme-gRNA-ribozyme) can include the following aspects. A sequence of twenty nucleotides can be prepared along with a sequence referred to as a scaffold, where the scaffold binds to the 3′ of a selected gRNA. Two ribozymes, including the hammerhead ribozyme (HH) and hepatitis delta virus ribozyme (HDR), can be connected to the two sides of the SgRNA, with the HH towards 5′ of the gRNA and the HDV towards the 3′ of the gRNA. Following transcription, the ribozymes function to cut two sides of the SgRNA (right and left), allowing assembly of the SgRNA.

Transfer of an assembled CRISPR construct into a cell by electroporation can include the following aspects. Electroporation techniques were used to transfer the plasmid and construct (donor gRNA and CAS9) into the Pichia cells, where an electrical pulse can be used that leads to introducing the DNA into the cell. For this purpose, electrocompetent cells can be produced by using a condensed protocol that includes a short and quick method in comparison with regular electroporation. The donor template can be linearized prior to transforming the plasmid into the cell by electroporation. A restriction enzyme can be used to cut the donor template plasmid out of the donor sequence region. For example, the donor template plasmid can be cut with SpeI enzyme to linearize the plasmid, after which the plasmid can be condensed and precipitated to produce a concentrated amount thereof (e.g., more than 1 ug per ul) by using absolute ethanol and sodium acetate (3M). Then, 1.5 microgram donor plasmid with 100 nanograms CAS9 and gRNA plasmid can be transformed into the electrocompetent cells by electroporation. To produce electrocompetent Pichia cells, a BEDS solution (Bicine, ethylene glycol, DMSO, and sorbitol) can be used. A MicroPulser™ from Bio-Rad (Hercules, California, USA) and a 2 mm cuvette with 1500 V and 25 uF can be used for the electroporation step. After electroporation, 1 mL of YPDS solution can be added and the cells can be incubated overnight at 30 degrees C. The next day the electroporated samples can be cultivated on YPDS media selecting for hygromycin antibiotic resistance. After 72 hours, transformed colonies appeared.

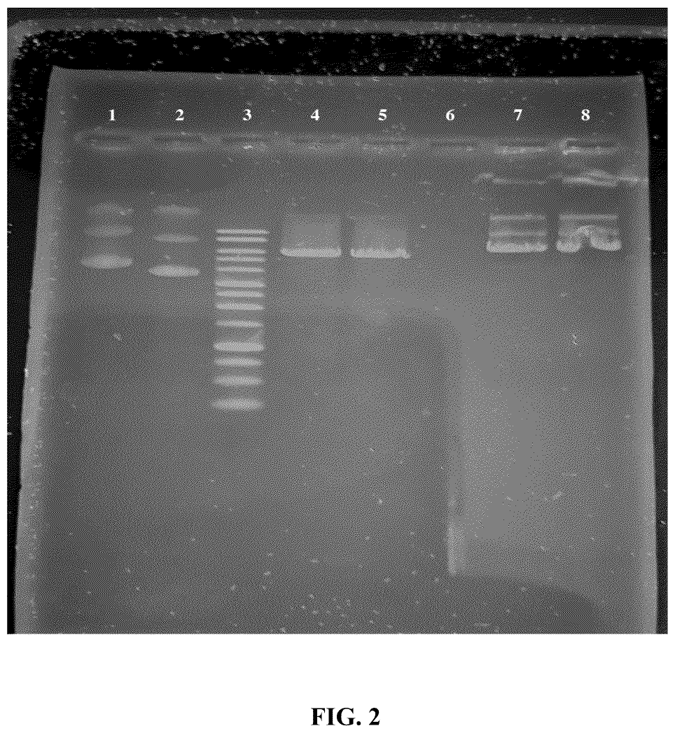

FIG. 2 demonstrates plasmid extraction and linearization, showing from left to right: (1) AOX1 plasmid extraction, (2) FLD1 plasmid extraction, (3) molecular marker, (4) linearized FLD1, and (5) linearized AOX1.

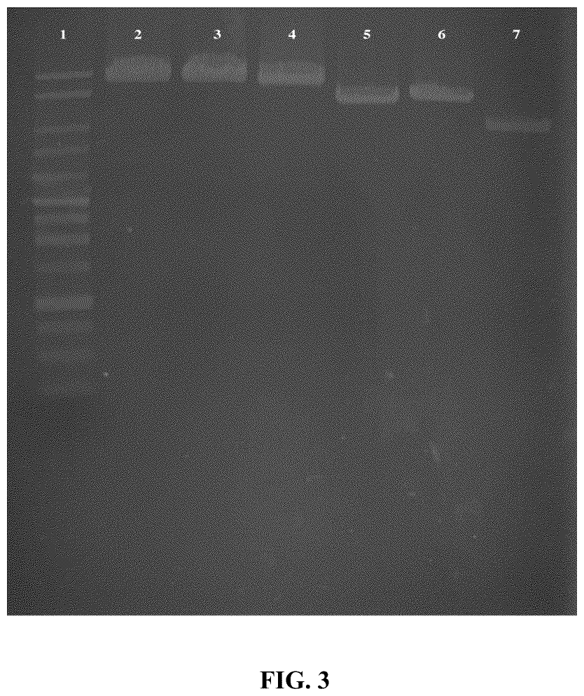

FIG. 3 demonstrates plasmid linearization by restriction enzymes, showing from left to right: (1) molecular marker, (2) FLD1 plasmid, (3) AOX1 plasmid, (4) GAP plasmid, (5) IDI-GPPS2 plasmid, (6) ThiM-MtIPK plasmid, and (7) NphB plasmid.

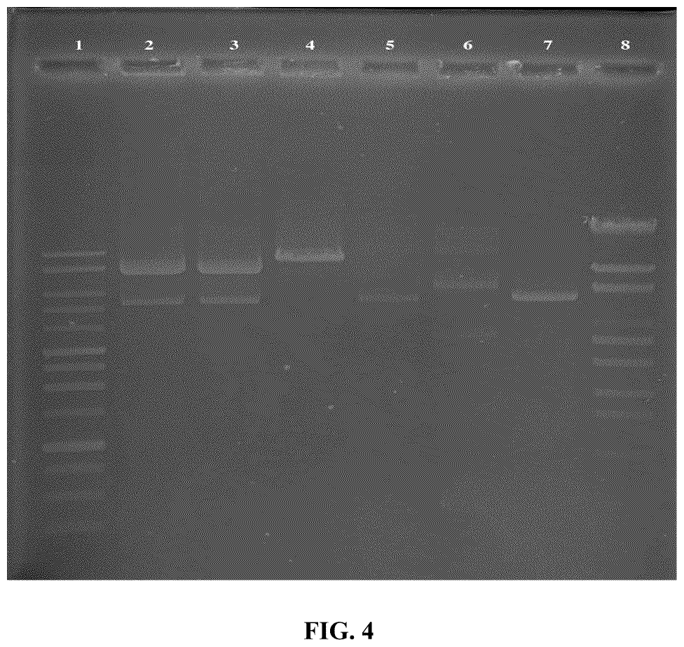

FIG. 4 demonstrates plasmid extraction and linearization, showing from left to right: (1) molecular marker, (2) & (3) AOX1 plasmid extraction, (4) is linearized AOX1 plasmid, (5) & (7) linearized NphB, and (6) is NphB plasmid circular.

The selection of transformants using selective media can include the following aspects. Seventy-two hours following electroporation, the transformed colonies can be sorted from the media. The colonies can include three groups: (1) colonies that received the CAS9 and gRNA plasmid but the CAS9 cannot cut the DNA (due to the low efficiency of gRNA); (2) transformed colonies that received gRNA and CAS9 plasmid but the HR process was not completed and the donor sequence could not introduce into the genome, but the NHJE pathway repaired the cutting area; and (3) transformed colonies which received gRNA and CAS9 plasmid, as well as donor plasmid, went through the HR process, where this group is preferred and was subjected to verification. It was found to be better to select a small colony and have these colonies cultivated in YPD medium, where after a couple of generations, they gradually lose their CAS9 plasmid. Following 18-24 hours of cultivation in YPD medium, the transformed yeast can be plated following centrifugation and verified by DNA techniques.

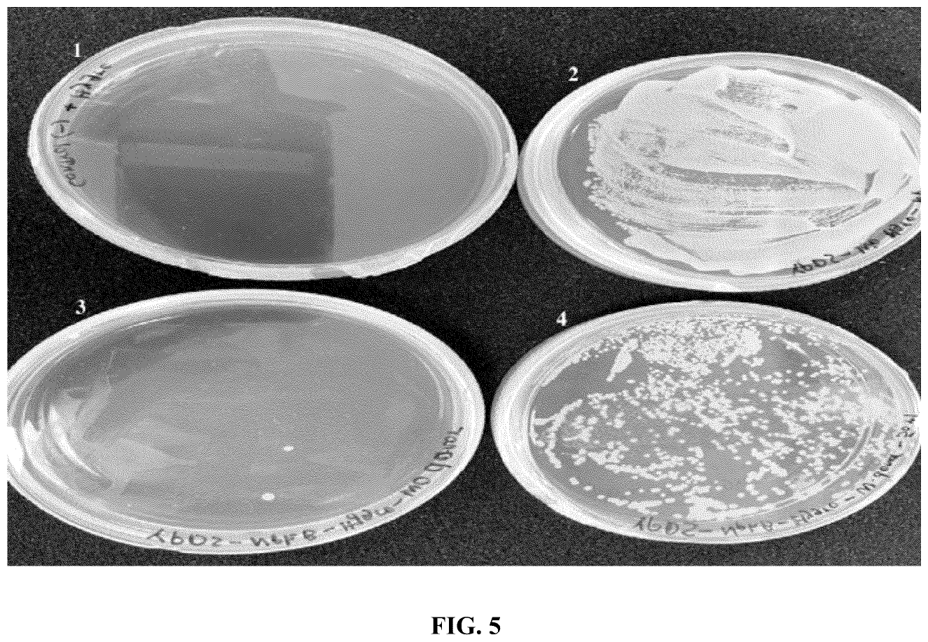

FIG. 5 demonstrates the cultivation of colonies and transformant colony verification, where: (1) negative control (without plasmid+with hygromycin); (2) growth medium without any antibiotics (to identify the viability of Pichia cells); (3) colonies with CRISPR construct and without donor template; and (4) colonies with the CRISPR construct and with donor template.

PCR verification of transformants can include the following aspects. Transformed colonies can be cultivated in medium without antibiotics and after 18-24 hours DNA can be extracted from the colonies. PCR amplification can include gene-specific primer binding to the respective gene introduced to the genome. The primers can be designed with Primer 3 online software and the designed primers can be checked with Oligo Analyzer software. Suitable primers and primer pairs that minimize self-complementarity and dimers can be selected. It can be desirable to have the forward primer bind to the construct (e.g., the gene of interest) and the reverse primer bind to the Pichia pastoris genome to prevent the production of a false-positive. After designing the primers, the primers can be synthesized (by Integrated DNA Technologies) and PCR amplification can be performed using a Promega kit. In this way, the CRISPR construct and the transformation efficiency can be optimized. After PCR amplification, the samples can be loaded onto an agarose gel and the presence of PCR product bands can be visualized using a UV transilluminator.

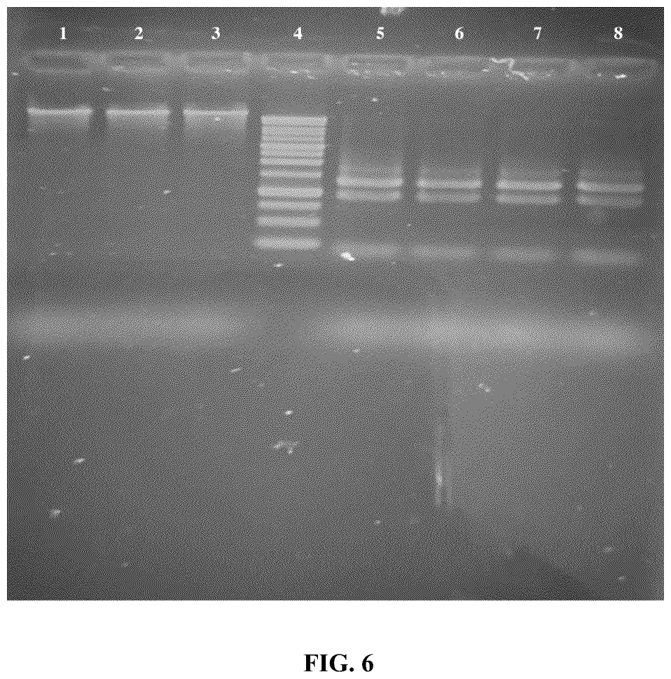

FIG. 6 demonstrates DNA and RNA extraction from colony samples. Lanes one through three are DNA extracted from colonies. Lane four is a molecular marker (1 Kb). Lanes 5 to 8 are RNA extracted from colonies used for real time PCR to check expression level and thereby determine if the promoter/terminator (transcription unit) is functional.

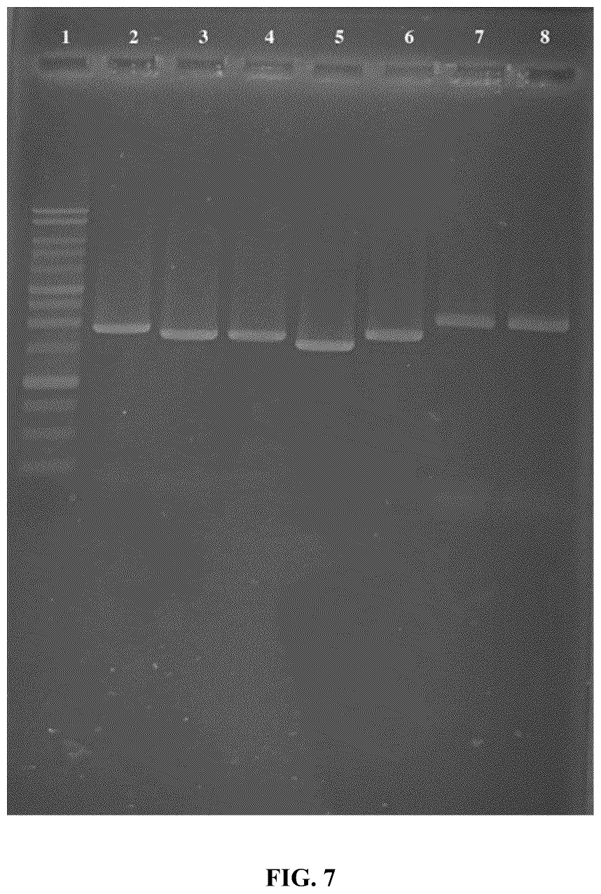

FIG. 7 demonstrates DNA verification using specific primers, where the lanes are as follows: (1) 1 Kb molecular marker; (2) primer specific for NphB (1750 bp); (3) primer specific for MtIPK (1590 bp); (4) primer specific for GPPS2 (1600 bp); (5) primer specific for NphB (1558 bp); (6) primer specific for IDI (1985 bp); and (7) & (8) are primers specific for ThiM (2018 bp).

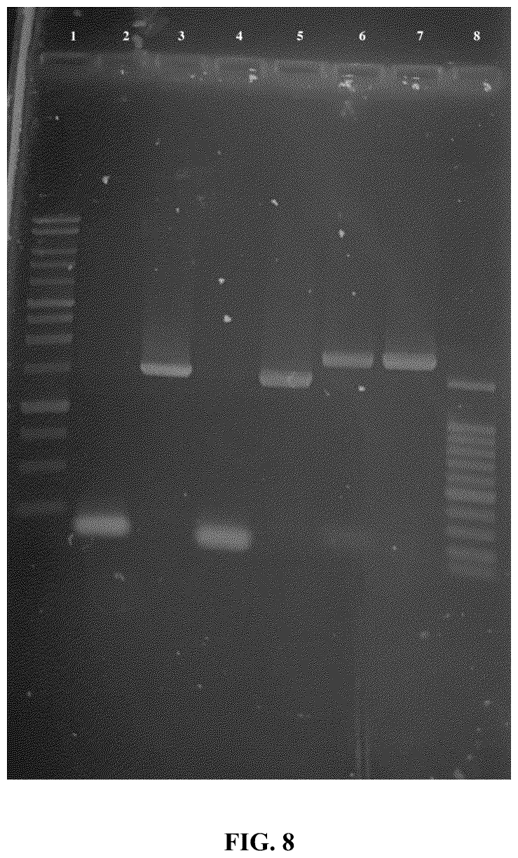

FIG. 8 demonstrates DNA verification by some specific primers, where the lanes are as follows: (1) 1 kb Molecular marker, (2) ΔKu70 with p1 primer, (3) NphB with p1 primer, (4) ΔKu70 with p2 primer, (5) NphB with p2 primer, (6) & (7) nontransgenic clones with p3 primer, which is designed from the Pichia genome, and (8) 100 bp molecular marker.

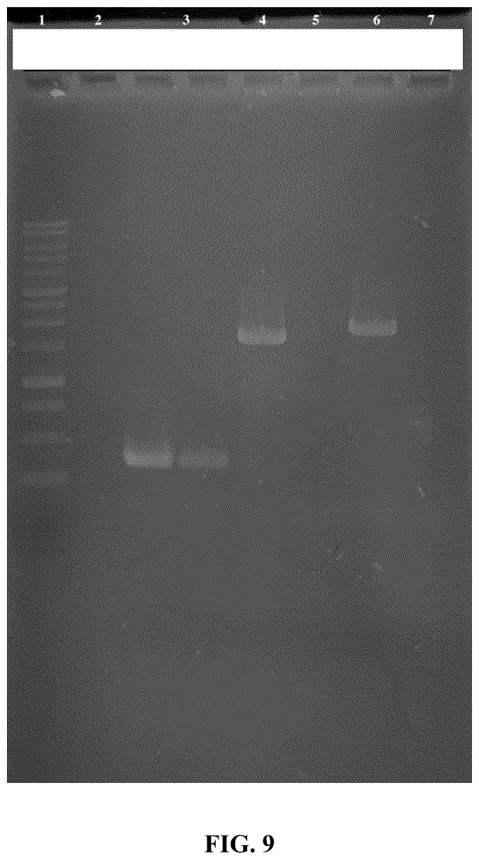

FIG. 9 demonstrates NphB verification with 3 primers, (1) molecular marker 1 kb, (2) ΔKu70 Strain with P1 primer, (3) transformed colony with P1 primer, (4) transformed colony with P2 primer, (5) ΔKu70 Strain with P2 Primer, (6) transformed colony with P3 primer, (7) ΔKu70 Strain with P3 primer.

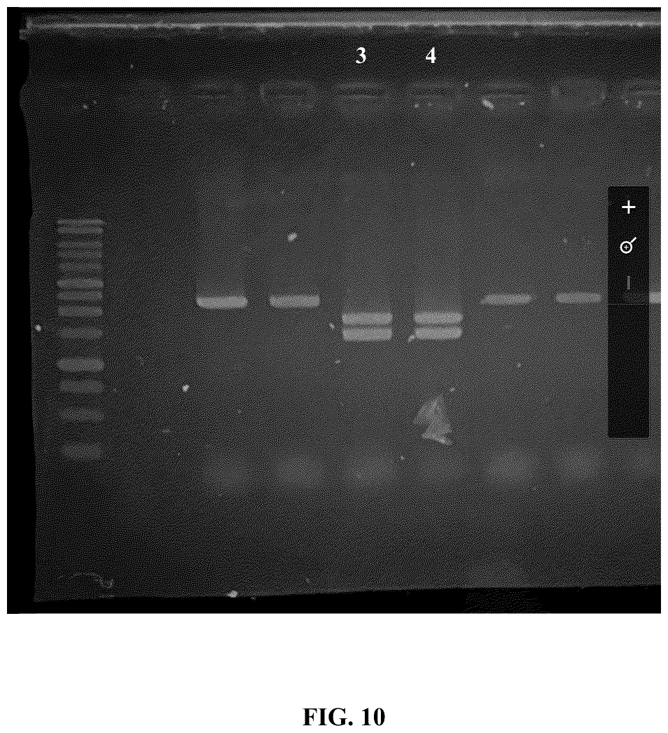

FIG. 10 demonstrates the verification of NphB gene with specific primers, where lanes 3 & 4 are from transformed colonies and the other lanes are from colonies that are not transformed.

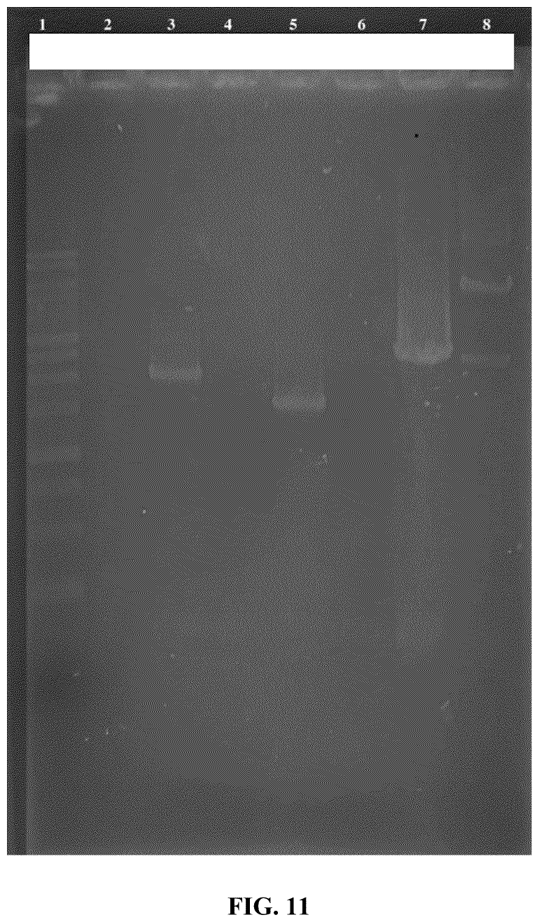

FIG. 11 depicts DNA verification for Thim and MtIPK genes. Lane 1 is a molecular marker (1 Kb). Lane two is a negative control. Lane three is ThiM-gene amplified with a single specific primer. Lane four Ku70 (strain) without the ThiM gene to act as a control. Lane five is the amplification of primer (MtIPK). Lane six is a KU-70 strain without MtIPK gene.

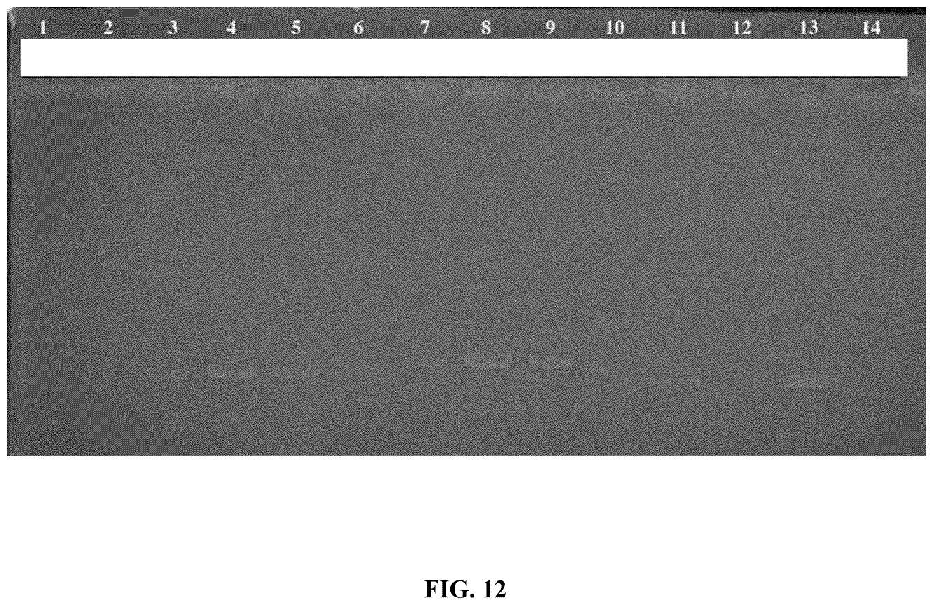

FIG. 12 depicts DNA Verification for NphB, IDI, and GPPS2 genes. Lane one is a molecular marker (1 Kb). Lane two is a negative control. Lanes three, four, and five are amplifications with ThiM primer. Lane six is Ku70 strain used as a negative control. Lanes seven, eight, and nine are amplifications with MtIPK primer. Lane ten is Ku70 strain negative control. Lanes eleven, twelve, and thirteen are amplifications of NphB primer (gene). Lane fourteen is Ku70 strain (negative control).

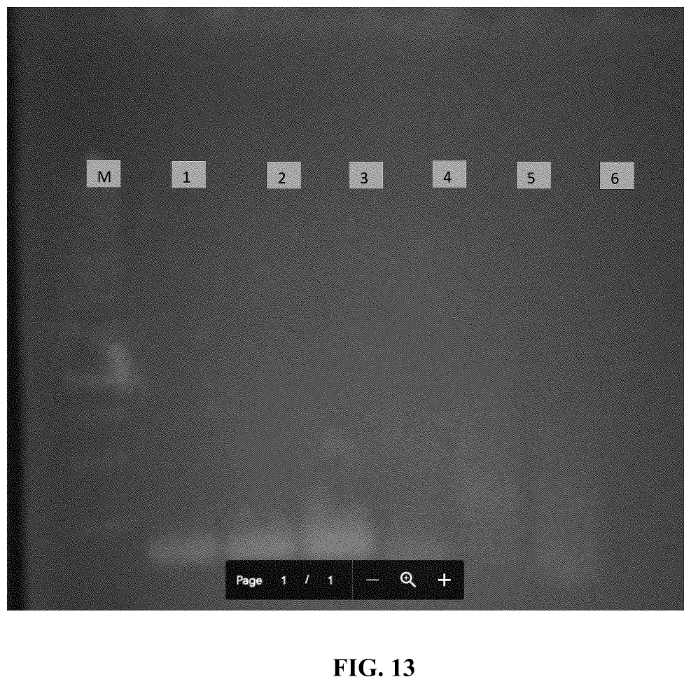

FIG. 13 depicts semi-quantitative RT-PCR for RNA verification of NphB, where (1) first-time course after induction (24 h), (2) second time course after induction (48 h), (3) third time course after induction (72 h), (4) before induction, (5) −Ku70 strain before induction, (6) −Ku70 strain after induction.

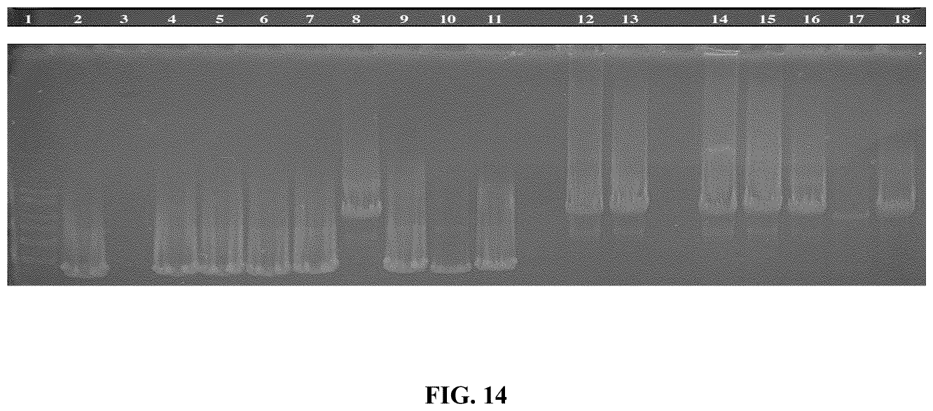

FIG. 14 depicts DNA verification for IDI and GPPS2 genes, where (1) molecular marker, (2) ΔKu70, (3) negative control, (4), (5), (6), (7), (9), (10) and (11) are colonies without gene insertion, (8), (12), (13), (14), (15), (16), and (18) are transformed colonies with IDI and GPPS2 gene insertions.

RNA verification by reverse transcription PCR (RT-PCR) can include the following aspects. After the transformed colonies containing the gene of interest are confirmed by PCR, the colonies can be verified at the RNA level to identify gene expression patterns. The samples including our genes of interest can be cultivated in a minimal medium called MGAS, which contains YNB, biotin, glycerol, and ammonium sulphate. After 24 hours, the yeast cells can be extracted by centrifuge and cultivated in an induction media including YNB, biotin, glycerol, and methylamine (as a nitrogen source). The methylamine in the induction media can induce the FLD1 promotor and lead to mRNA synthesis. RNA samples can be extracted using the Zymo research RNA kit in different time courses after induction (e.g., 0, 24 h, 48 h, 72 h, 96 h). cDNA can be synthesized from RNA using the cDNA synthesis Qiagen kit. Furthermore, RT-PCR can be performed using the primers designed using primer 3 online software. The resulting amplicon can be expected to be 50 to 200 base pairs with the primers designed from the CDS of the gene.

RT-PCR can be performed using the Qiagen QuantiNova Cyber green RT-PCR kit (Qiagen, Germantown, MD, USA), where GAPDH can be used as a positive internal control and GS115-Ku70 can be used as a negative control. After sequentially performing RT-PCR for each sequential transformation of the five subject genes involved in CBGA biosynthesis, RT-PCR can be performed for all five of the genes in the final recombinant organism. Certain embodiments of the present technology produced interesting results. After induction with methylamine, the gene expression rate increased for the five genes, and after 48 hours of induction the gene expression rate was suddenly reduced. By increasing methylamine concentration, the cells can be under metabolic pressure (metabolic burden), therefore the transcription machinery can be under high pressure which subsequently leads to a gene expression decrease. To resolve this issue, the conditions can be optimized in order to decrease the metabolic burden. Three different strategies can be employed, including (1) decreasing the temperature to 15 degrees C., (2) decreasing the inducer (methylamine) concentration, and (3) increasing the biomass in comparison to medium volume. These three strategies can be performed, and samples obtained in different time courses, (e.g., 0, 24 h, 48 h, 72 h, and 96 h) to extract RNA for RT-PCR amplification.

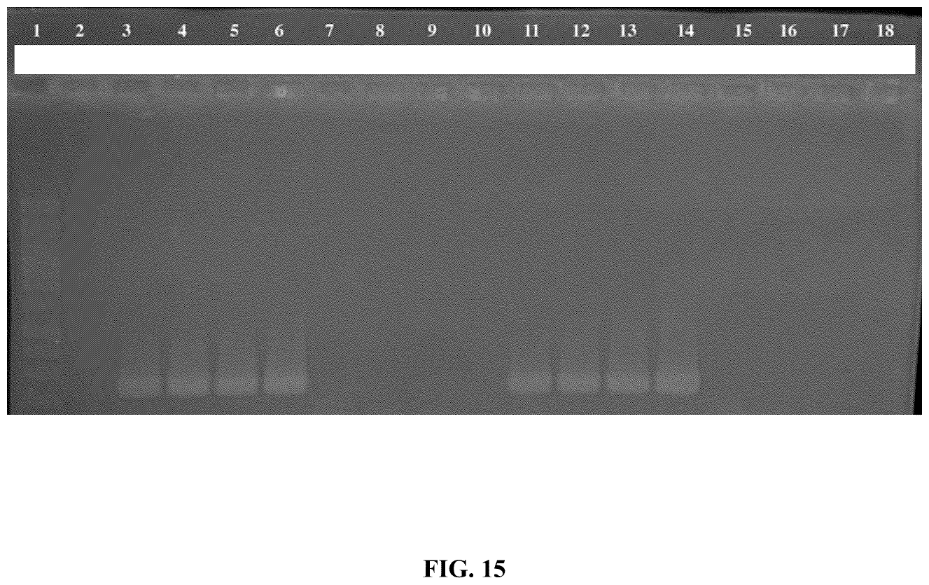

FIG. 15 depicts RNA verification with semiquantitative RT-PCR for IDI & GPPS2 genes. Lane one is a molecular marker (1 Kb). Lane two is a negative control. Lane three is RNA verification for specific IDI-primer within the first time course post-induction. Lane four is RNA verification for specific IDI-primer within the second time course post-induction. Lane five is RNA verification for specific IDI-primer within the third time course post-induction. Lane six is RNA verification for specific IDI-primer within the fourth time course post-induction. Lane seven is RNA verification for Ku70 strain, with IDI-primer at first time course. Lane eight is RNA verification for Ku70 strain, with IDI-primer at second time course. Lane nine is RNA verification for Ku70 strain, with IDI-primer at third time course. Lane ten is RNA verification for Ku70 strain, with IDI-primer at fourth time course. Lane eleven is RNA verification GPPS specific primer within the first time course. Lane twelve is RNA verification GPPS specific primer within the second time course. Lane thirteen is RNA verification GPPS specific primer within the third time course. Lane fourteen is RNA verification GPPS specific primer within the fourth time course. Lane fifteen is RNA verification for Ku70 strain with GPPS-primer first time course. Lane sixteen is RNA verification for Ku70 strain with GPPS-primer second time course. Lane seventeen is RNA verification for Ku70 strain with GPPS-primer third time course. Lane eighteen is RNA verification for Ku70 strain with GPPS-primer fourth time course.

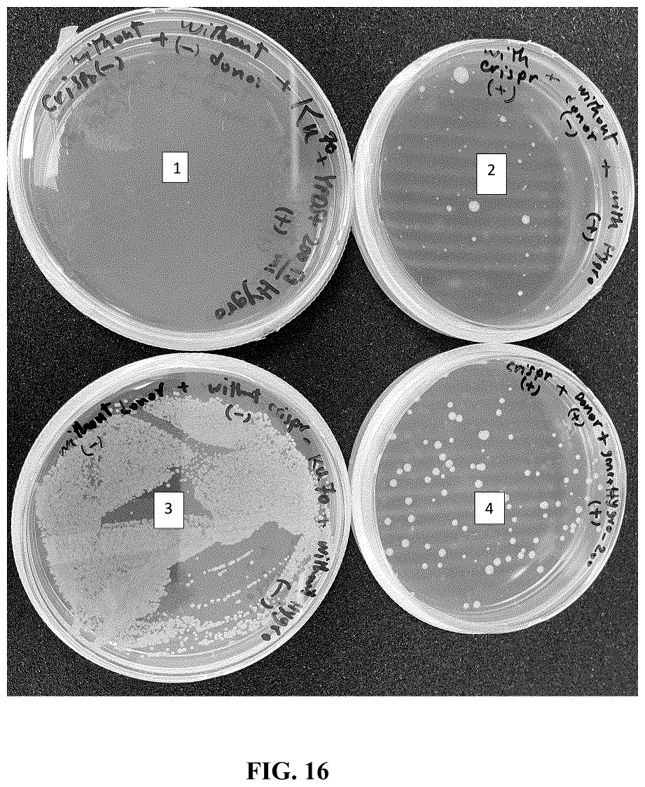

FIG. 16 depicts CRISPR colonies in YPDS medium with hygromycin antibiotics, where (1) negative control without any CRISPR construct in the 200 ug/ml hygromycin, (2) CRISPR construct without donor template in the 200 ug/ml hygromycin, (3) positive control without CRISPR construct & hygromycin, (4) CRISPR construct with donor template in the 200 ug/ml hygromycin.

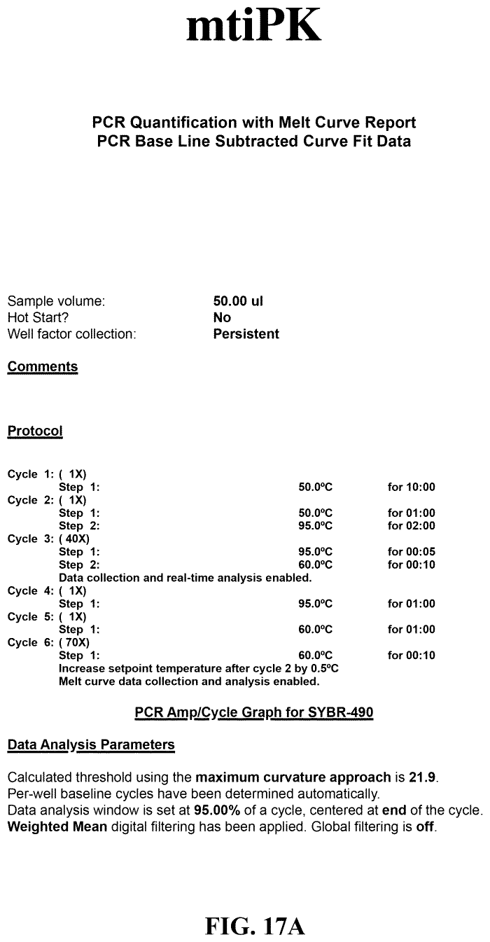

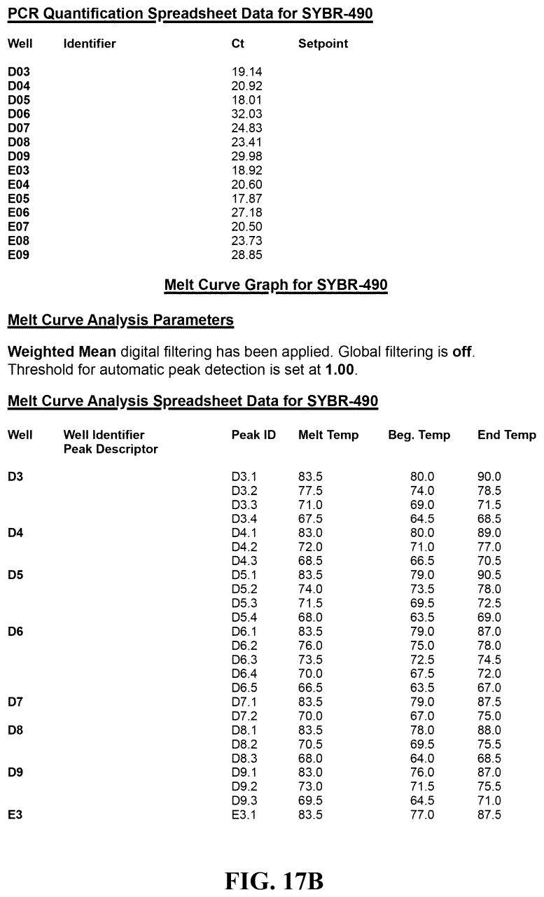

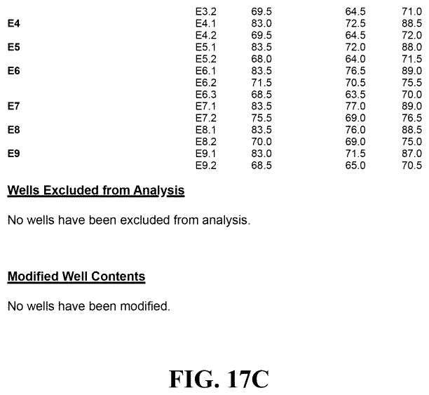

FIGS. 17A, 17B, and 17C depict MtIPK PCR quantification with a melt curve report and PCR baseline subtracted curve fit data.

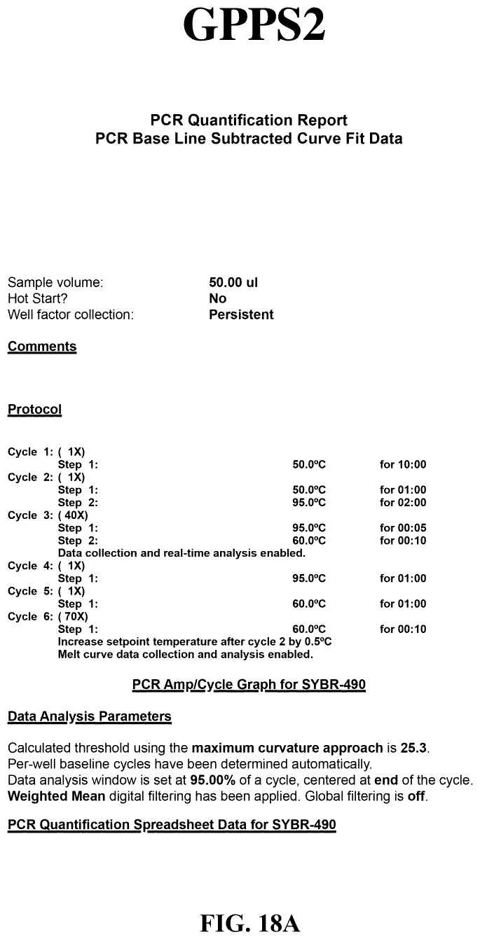

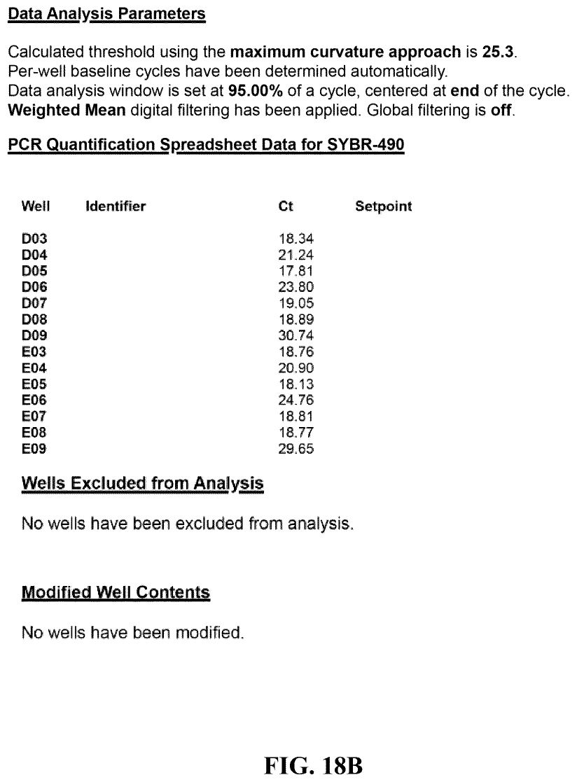

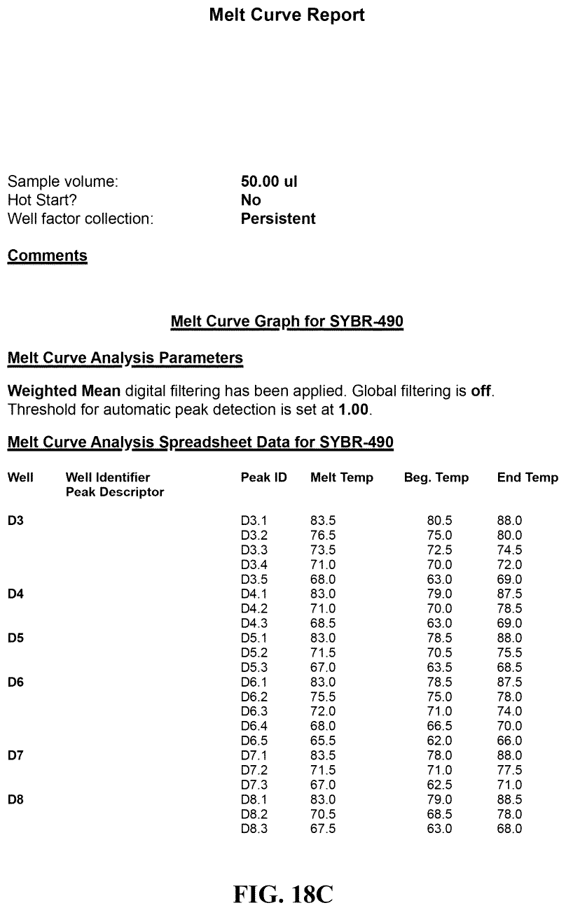

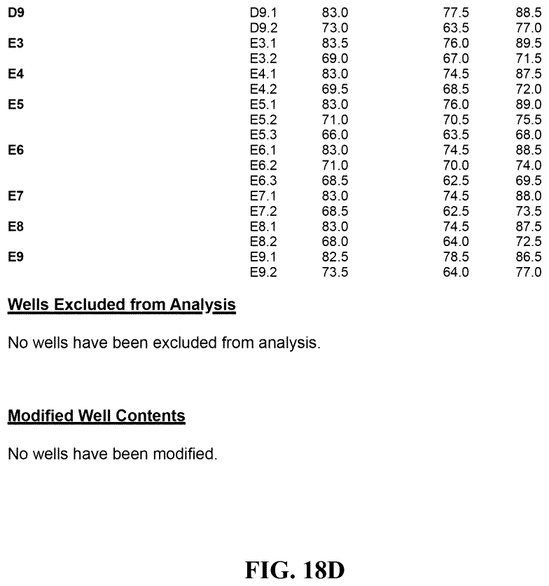

FIGS. 18A, 18B, 18C, and 18D depict GPPS PCR quantification report and PCR baseline subtracted curve fit data.

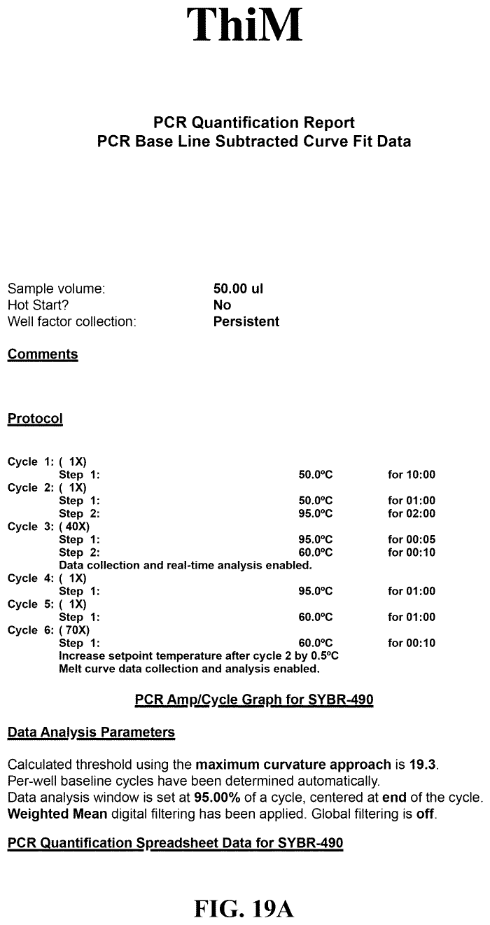

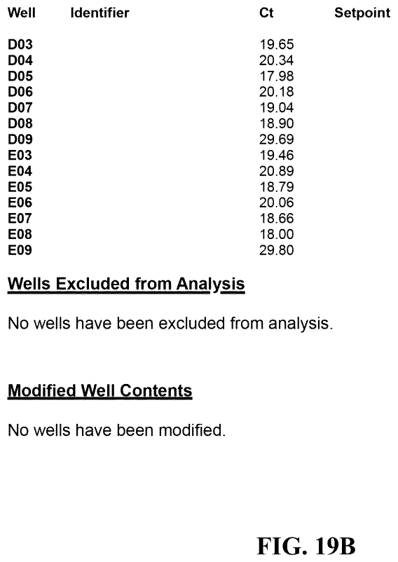

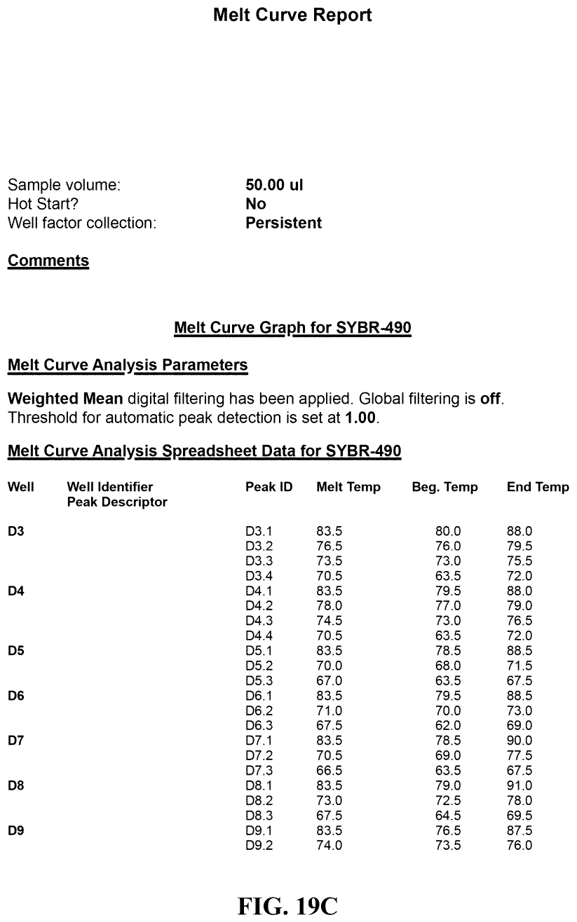

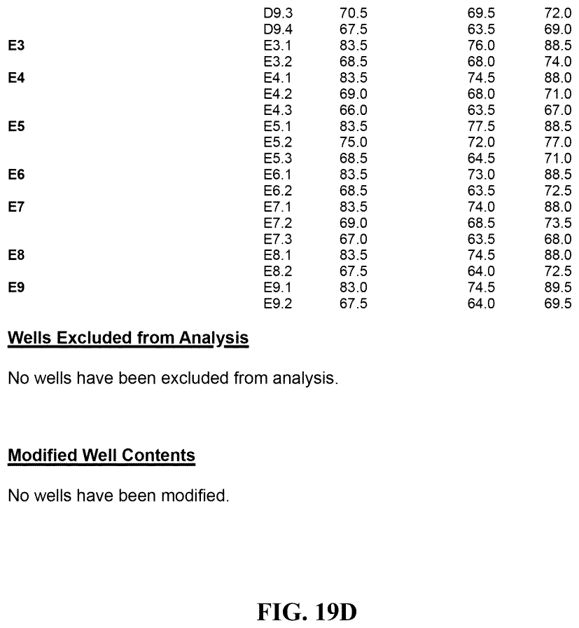

FIGS. 19A, 19B, 19C, and 19D depict ThiM PCR quantification report and PCR baseline subtracted curve fit data.

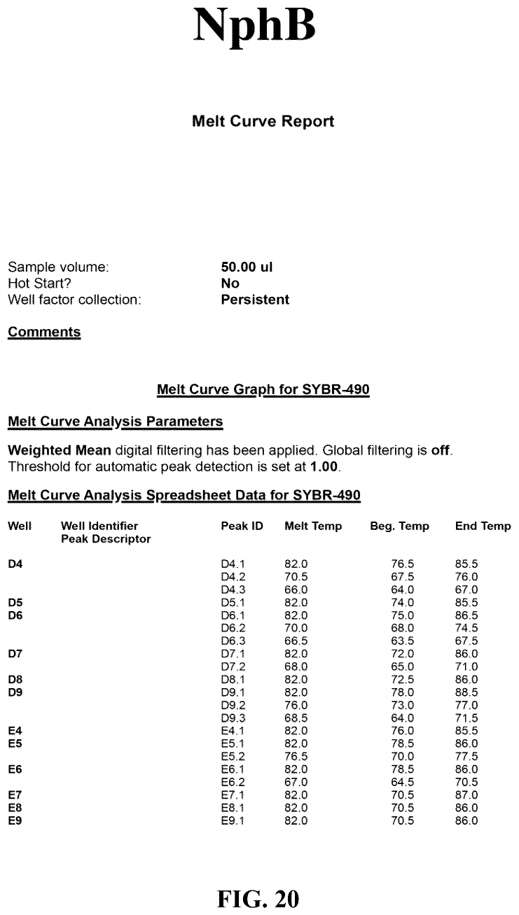

FIG. 20 depicts NphB PCR quantification report, where no wells are excluded from analysis and no wells are modified.

Protein verification by SDS-PAGE and western blotting techniques can include the following aspects. Once the genes are verified by PCR and the expression rate is identified by RT-PCR, the expressed proteins can be validated by using the SDS-PAGE and western blotting technique. For this reason, certain protein tags (e.g., histidine tags) can be added to the 5′ or 3′ ends of the genes. Then, by using the anti-tag (e.g., anti-his tag) the desired protein can be identified using the western blot technique. The tag used in certain embodiments included a 6× histidine that can be introduced 3′ of the desired genes prior to stop codons. Also, the polylinker sequence (Gly-Gly-Ser-Gly) can be used between the his-tag and the gene sequence. In order to perform the SDS-PAGE and western blot, proteins can be extracted from different time courses after induction by using a yeast buster solution from Novagen. Protease inhibitors can be added to the yeast cell lysate to prevent protein degradation. Protein samples can be heated up to 100 degrees C. for five minutes and loaded on the polyacrylamide gels. Protein bands can be observed and transferred to a PVDF membrane by electroblotting and can be identified by using anti-his-tag bound to a colorimetric indicator. Suitable kits and corresponding protocols include those available from Nanoprobes Inc. (Yaphank, NY, USA).

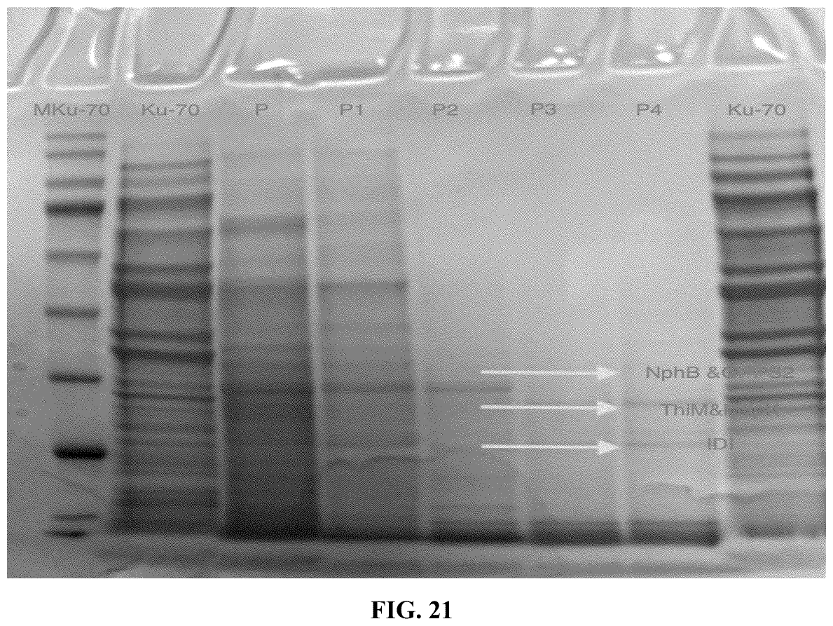

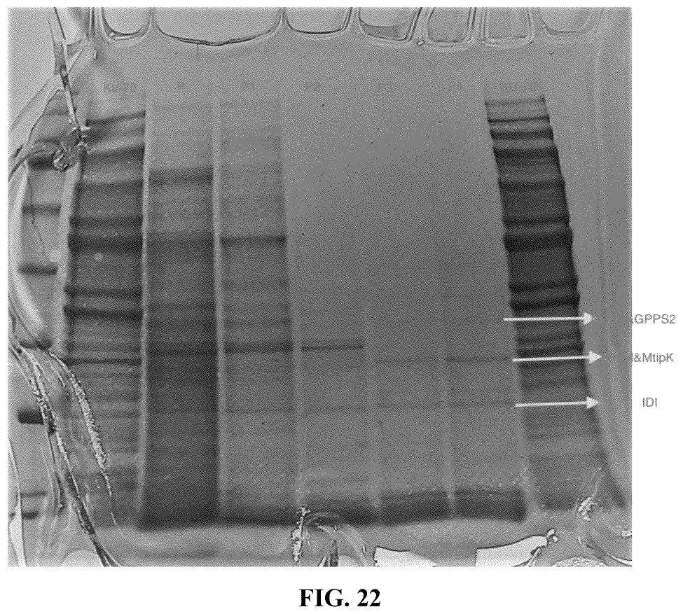

FIGS. 21 and 22 depict protein verification with SDS-PAGE, where (M) molecular marker for protein, (KU-70) negative control, (P) sample before induction, (P1) first-time course after induction, (P2) second time course after induction, (P3) third time course after induction, (P4) fourth time course after induction.

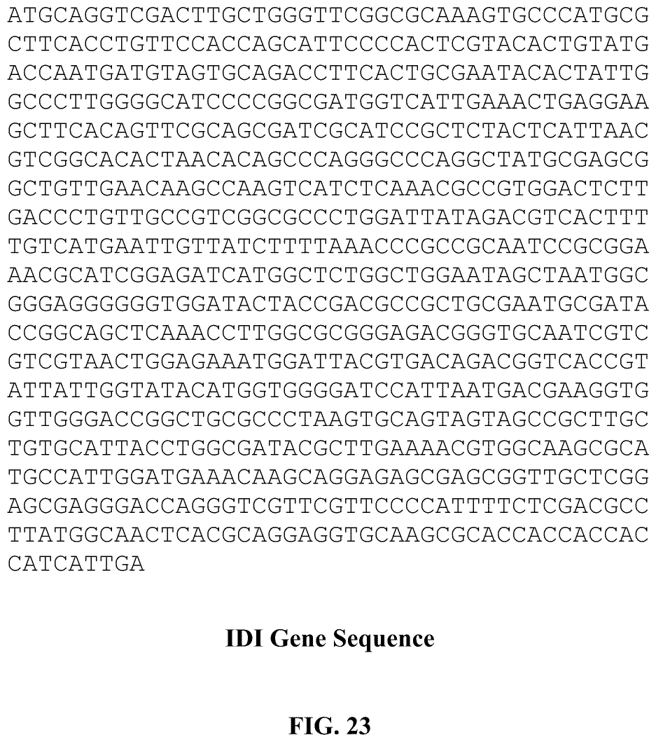

The IDI gene sequence after codon optimization for Pichia is shown in FIG. 23 and provided as SEQ ID NO. 1.

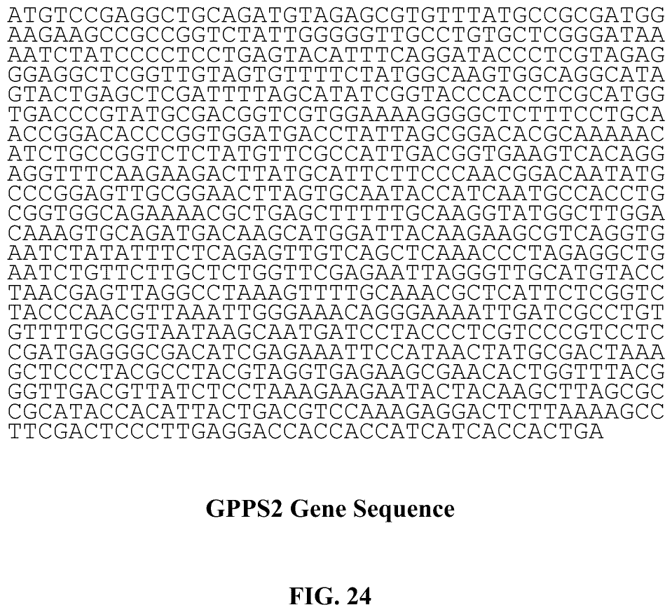

The GPPS2 gene sequence is shown in FIG. 24 and provided as SEQ ID NO. 2.

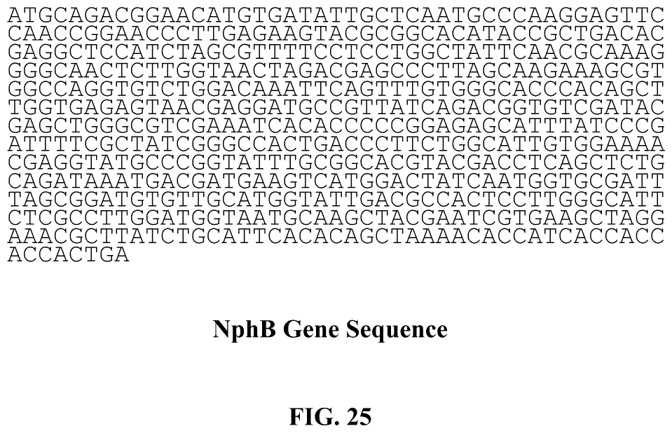

The NphB gene sequence is shown in FIG. 25 and provided as SEQ ID NO. 3.

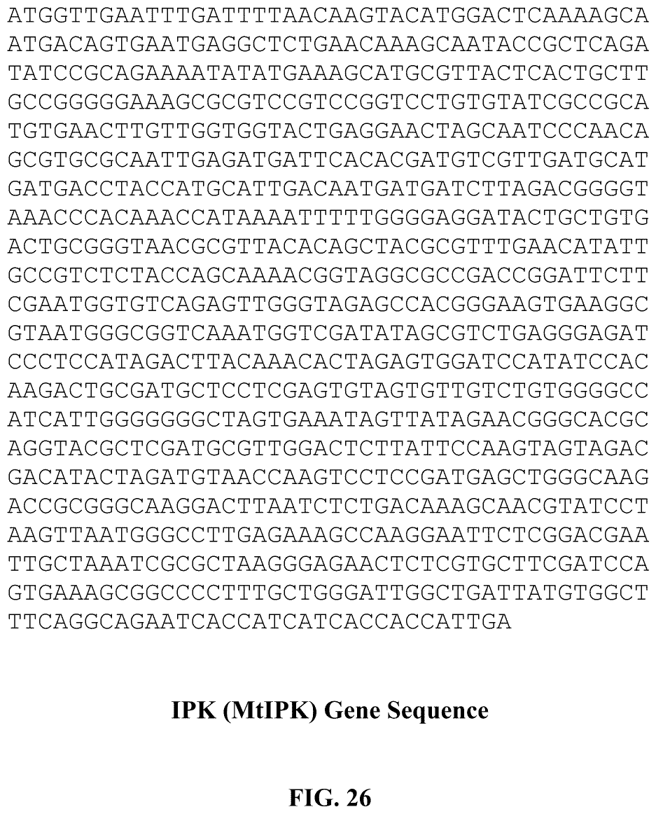

The MtIPK gene sequence is shown in FIG. 26 and provided as SEQ ID NO. 4.

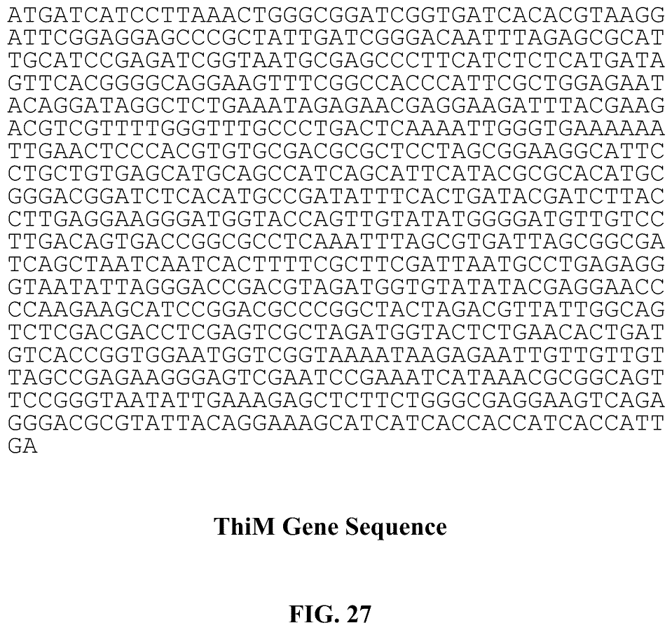

The ThiM gene sequence is shown in FIG. 27 and provided as SEQ ID NO. 5.

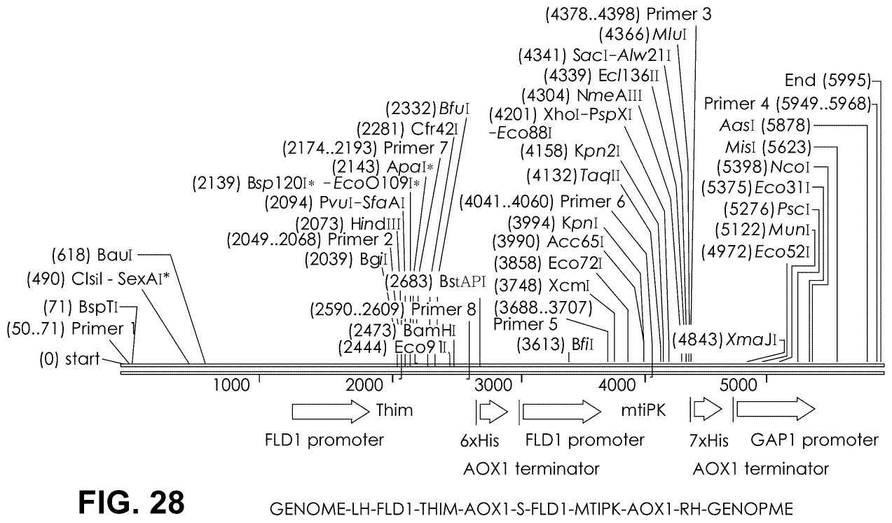

FIG. 28 depicts a ThiM-MtIPK recombinant construct, where associated primers related thereto are provided as follows:

ThiM
Primer1:
(SEQ ID NO. 6)
5′-GGATGCAATGCCTGTTCTTTTC-3′
Primer2:
(SEQ ID NO. 7)
5′-AGTTTCAATGACCATCGCCG-3′
mtipK
Primer1:
(SEQ ID NO. 8)
5′-GGAAAGCATCATCACCACCAT-3′
Primer2:
(SEQ ID NO. 9)
5′-AACCCAAAACACCCTTGAGC-3′
mtipK-RNA
Primer1:
(SEQ ID NO. 10)
5′-GTAATGCGAGCCCTTCATC-3′
Primer2:
(SEQ ID NO. 11)
5′-CGCCGCTAATCACGCTAAAT-3′
ThiM-RNA
Primer1:
(SEQ ID NO. 12)
5′-AAGTCATCTCAAACGCCGTG-3′
Primer2:
(SEQ ID NO. 13)
5′-CTCGCTCTCCTGCTTGTTTC-3′
Real-Time NphB-RNA
Primer1:
(SEQ ID NO. 14)
5′-ACGTAGGTGAGAAGCGAACA-3′
Primer2:
(SEQ ID NO. 15)
5′-TCAGTGGTGATGATGGTGGT-3′
Real-Time ThiM-RNA
Primer1:
(SEQ ID NO. 16)
5′-GGTCGTTCGTTCCCCATTTT-3′
Primer2:
(SEQ ID NO. 17)
5′-CAATGATGGTGGTGGTGGTG-3′
Real-Time MtipK-RNA
Primer1:
(SEQ ID NO. 18)
5-TCATCCTTAAACTGGGCGGA-3′
Primer2:
(SEQ ID NO. 19)
5-GATGAAGGGCTCGCATTACC-3′
Real-Time IDI-RNA
Primer1:
(SEQ ID NO. 20)
5′-GGCATTGTGGAAAACGAGGT-3′
Primer2:
(SEQ ID NO. 21)
5′-CCGCTAAATCGCACCATTGA-3′
Real-Time GPPS2-RNA
Primer1:
(SEQ ID NO. 22)
5′-CGTCTCTACCAGCAAAACGG-3′
Primer2:
(SEQ ID NO. 23)
5′-CATTACGCCTTCACTTCCCG-3′
GAPDH
Primer1:
(SEQ ID NO. 24)
5′-AGAGGTGGTAGAACGGCTTC-3′
Primer2:
(SEQ ID NO. 25)
5′-CGGTCAAGTCAACAACGGAG-3′

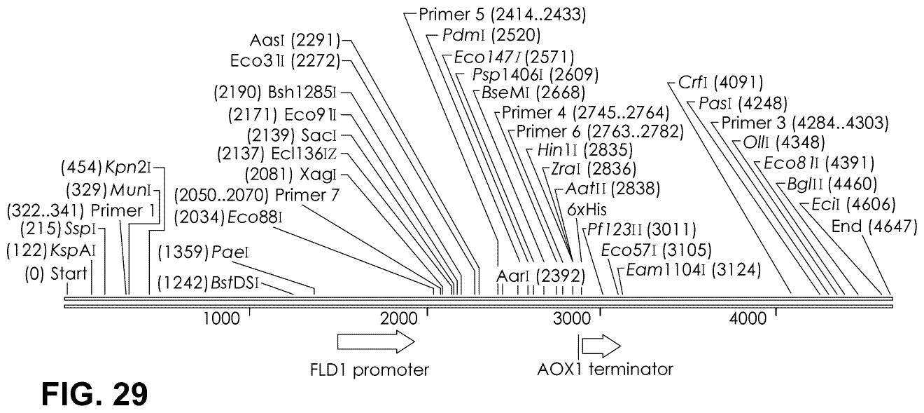

FIG. 29 depicts a NphB recombinant construct, where associated primers related thereto are provided as follows:

NphB
Primer1:
(SEQ ID NO. 26)
5′-ACACCTACAATTGGAGCCCA-3′
Primer2:
(SEQ ID NO. 27)
5′-TGAAATGTACTCAGGAGGGGA-3′
NphB-RNA
Primer1:
(SEQ ID NO. 28)
5′-GCAAGGTATGGCTTGGACAA-3′
Primer2:
(SEQ ID NO. 29)
5′-CGTCAACCCGTAAACCAGTG-3′
NphB2-R
Primer1:
(SEQ ID NO. 30)
5′-ACGTAGGTGAGAAGCGAACA-3′
Primer2:
(SEQ ID NO. 31)
5′-CCTTGCCCTTACAACGGAAA-3′

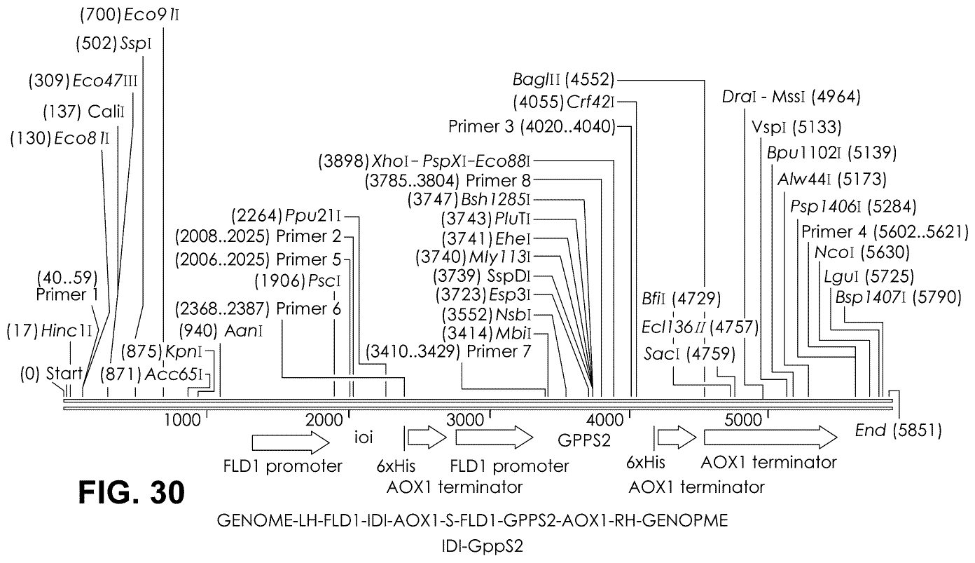

FIG. 30 depicts an IDI-GPPS2 recombinant construct, where associated primers related thereto are provided as follows:

GPPS2-RNA
Primer1:
(SEQ ID NO. 32)
5′-TACCGCTCAGTTATCCGCAG-3′
Primer2:
(SEQ ID NO. 33)
5′-CATTACGCCTTCACTTCCCG-3′
IDI-RNA
Primer1:
(SEQ ID NO. 34)
5′-TGGCTATTCAACGCAAAGGG-3′
Primer2:
(SEQ ID NO. 35)
5′-GCATTACCATCCAAGGCGAG-3′
IDI
Primer1:
(SEQ ID NO. 36)
5′-GGCTTGAGTCCTGGCTACAT-3′
Primer2:
(SEQ ID NO. 37)
5′-CCCTTTGCGTTGAATAGC-3′
GPPS2
Primer1:
(SEQ ID NO. 38)
5′-TGTAACCAAGTCCTCCGATGA-3′
Primer2:
(SEQ ID NO. 39)
5-GGTTGTTCTCACCTGCTTCG-3′

Although use of a CRISPR construct is described to knock-in the selected genes into the Pichia genome, every CRISPR construct can have certain specific elements to introduce into the genome. So, by changing the genes, some construct elements can be changed, which can also depend on the desired location in which to introduce the genes. One skilled in the art can select a predetermined location into which to introduce the genes and can ascertain the location, expression, and activity thereof in accordance with the present technology.

The present technology therefore allows production of cannabinoids, such as cannabigerolic acid (CBGA), in a recombinant microorganism, such as Pichia pastoris. Production of CBGA affords production of many cannabinoids such as THCA, CBDA, CBCA, and so on. CBGA can serve as a precursor in producing many different cannabinoids that can be further processed, chemically modified, and combined with other compounds. The present technology accordingly provides a detailed way to make and use a recombinant microorganism to produce a cannabinoid. The expression of the enzymes in the recombinant construct (e.g., geranyl diphosphate synthase (GPPS2), the isopentenyl diphosphate isomerase (IDI), the isopentenyl phosphate kinase (IPK), and/or the 5-(hydroxyethyl)-methyl thiazole kinase (ThiM)) can each be ascertained as shown herein using RT-PCR, the enzymatic activities and corresponding reaction products produced thereby can be ascertained using standard analytical chemical techniques known to the skilled artisan, and verification of the biosynthetic output can be demonstrated as detailed herein. Based upon this detailed description, it is submitted that one of ordinary skill in the art can make and use the present technology without undue experimentation and accordingly a biological deposit is believed to not be necessary, as the assembly and screening assays provided herein provide a complete and verifiable roadmap to replicate the present technology. However, should a biological deposit of a recombinant organism configured to produce a cannabinoid in accordance with the present technology be deemed necessary, such a deposit can be made and the addition of information designating the depository, accession number, and deposit date of the deposited cell line in ATCC can be provided.

The enzymes provided herein, along with expression and activity thereof, can be readily characterized as described herein and understood by one skilled in the art. It should be appreciated that variants are contemplated by the present technology, where the expression and activity of such variants can be confirmed using the methods provided herein and understood by one skilled in the art. It is therefore possible to have nucleotide sequence variants of the subject genes and amino acid variants of the resultant proteins/enzymes based upon the degeneracy of the genetic code and/or based upon predetermined conservative amino acid substitutions that can be confirmed in accordance with the methods provided by the present disclosure to retain the desired expression level as well as enzymatic activity thereof. In this way, nucleotide sequence (and resultant amino acid sequence) can vary from the specific sequences listed herein. Embodiments include variants having from 75% up to 99.9% sequence identity to the specific sequences listed herein, where certain examples include sequence identities of 75%, 80%, 85%, 90%, 95%, 96%, 97%, 98%, and 99%.

Nucleotide or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are known to those skilled in the art. Publicly available computer software such as BLAST, BLAST2, ALIGN2 or Megalign (DNASTAR) software can be used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=(X/Y)(100), where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which an amino acid that is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell; e.g., P. pastoris.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A recombinant organism configured to produce a cannabinoid, comprising:
a eukaryotic microorganism expressing a recombinant construct including a geranyl diphosphate synthase (GPPS2), an isopentenyl diphosphate isomerase (IDI), an isopentenyl phosphate kinase (IPK), and a 5-(hydroxyethyl)-methyl thiazole kinase (ThiM).

2. The recombinant organism of claim 1, wherein the eukaryotic organism includes a member of the fungus kingdom.

3. The recombinant organism of claim 1, wherein the eukaryotic organism includes a methylotrophic yeast.

4. The recombinant organism of claim 1, wherein the eukaryotic organism includes Pichia pastoris.

5. The recombinant organism of claim 1, wherein the geranyl diphosphate synthase (GPPS2) is derived from Abies grandis, the isopentenyl diphosphate isomerase (IDI) is derived from E. coli, the isopentenyl phosphate kinase (IPK) is derived from Methanothermobacter, and the 5-(hydroxyethyl)-methyl thiazole kinase (ThiM) is derived from E. coli.

6. The recombinant organism of claim 1, wherein expression of the geranyl diphosphate synthase (GPPS2) includes expression of a gene having at least 75% homology to SEQ ID NO. 2, expression of the isopentenyl diphosphate isomerase (IDI) includes expression of a gene having at least 75% homology to SEQ ID NO. 1, expression of the isopentenyl phosphate kinase (IPK) includes expression a gene having at least 75% homology to SEQ ID NO. 4, and expression of the 5-(hydroxyethyl)-methyl thiazole kinase (ThiM) includes expression a gene having at least 75% homology to SEQ ID NO. 5.

7. The recombinant organism of claim 1, wherein the recombinant construct further includes prenyltransferase (NphB).

8. The recombinant organism of claim 7, wherein the prenyltransferase (NphB) is derived from Streptomyces.

9. The recombinant organism of claim 7, wherein expression of the prenyltransferase (NphB) includes expression a gene having at least 75% homology to SEQ ID NO. 3.

10. The recombinant organism of claim 1, wherein the recombinant construct is incorporated into the genomic DNA of the recombinant organism.

11. The recombinant organism of claim 1, wherein the recombinant organism includes a Ku70 gene knockout.

12. The recombinant organism of claim 11, wherein the Ku70 gene knockout includes a selectable marker.

13. A recombinant organism configured to produce a cannabinoid, comprising:
a eukaryotic microorganism expressing a recombinant construct including a geranyl diphosphate synthase (GPPS2), an isopentenyl diphosphate isomerase (IDI), an isopentenyl phosphate kinase (IPK), and a 5-(hydroxyethyl)-methyl thiazole kinase (ThiM);
wherein:
the eukaryotic organism includes Pichia pastoris;
the geranyl diphosphate synthase (GPPS2) is derived from Abies grandis, the isopentenyl diphosphate isomerase (IDI) is derived from E. coli, the isopentenyl phosphate kinase (IPK) is derived from Methanothermobacter, and the 5-(hydroxyethyl)-methyl thiazole kinase (ThiM) is derived from E. coli;
the recombinant construct further includes prenyltransferase (NphB) derived from Streptomyces;
the recombinant construct is incorporated into the genomic DNA of the recombinant organism; and
the recombinant organism includes a Ku70 gene knockout.

14. A biosynthetic system for producing a cannabinoid, comprising:
a bioreactor;
the recombinant organism configured to produce the cannabinoid of claim 1; and
a growth medium for the recombinant organism.

15. The biosynthetic system for producing a cannabinoid of claim 14, where the growth medium for the recombinant organism includes prenol.

16. The biosynthetic system for producing a cannabinoid of claim 14, where the growth medium for the recombinant organism includes olivetolic acid.

17. A method of making a cannabinoid, comprising:
growing the recombinant organism configured to produce the cannabinoid of claim 1 in a growth medium.

18. The method of claim 17, further comprising:
adding prenol to the growth medium.

19. The method of claim 17, further comprising:
adding olivetolic acid to the growth medium.

20. The method of claim 17, further comprising:
isolating cannabigerolic acid following the growing step.
